(19) United States
(12) Patent Application Publication
(10) Pub. No.: US 2024/0199566 A1
(43) Pub. Date: Jun. 20, 2024

(54) CATALYTIC SYNTHESIS OF DELTA-VALEROLACTONE (DVL) FROM FURFURAL-DERIVED 2-HYDROXYTETRAHYDROPYRAN (HTHP)

(71) Applicant: Wisconsin Alumni Research Foundation, Madison, WI (US)

(72) Inventors: George Huber, Middleton, WI (US); Raka Dastidar, Madison, WI (US); Sam Johnstone, Madison, WI (US); Daniel McClelland, Fitchburg, WI (US)

(73) Assignee: Wisconsin Alumni Research Foundation, Madison, WI (US)

(21) Appl. No.: 18/510,856

(22) Filed: Nov. 16, 2023

Related U.S. Application Data
(60) Provisional application No. 63/425,875, filed on Nov. 16, 2022.

(51) Int. Cl. C07D 309/12; B01J 21/04; B01J 21/08; B01J 21/10; B01J 23/42; B01J 23/44; B01J 23/72; B01J 23/755

(52) U.S. Cl. CPC C07D 309/12; B01J 21/04; B01J 21/08; B01J 21/10; B01J 23/42; B01J 23/44; B01J 23/72; B01J 23/755

(57) ABSTRACT

A method to make delta-valerolactone (“DVL”) by dehydrogenating 2-hydroxytetrahydropyran (“HTHP”). The HTHP is contacted with a supported-metal catalyst for a time, at a temperature, and at a pressure wherein at least a portion of the HTHP is converted to DVL via dehydrogenation of the HTHP. The HTHP may be derived from biomass. Thus, the method yields DVL without requiring a petroleum-based feedstock.

### CROSS-REFERENCE TO RELATED APPLICATIONS

Priority is hereby claimed to provisional application Ser. No. 63/425,875, filed Nov. 16, 2022, which is incorporated herein by reference.

### FEDERAL FUNDING STATEMENT

This invention was made with government support under DE-EE0009305 awarded by the US Department of Energy. The government has certain rights in the invention.

### BACKGROUND

Delta-valerolactone (3-valerolactone; “DVL”) is a five-carbon cyclic ester that can undergo ring opening polymerization to produce aliphatic polyesters with excellent mechanical and biocompatible properties, making is useful for tissue engineering, drug delivery and surgical applications:

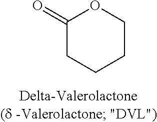

DVL is conventionally produced by dehydrogenating petroleum-derived 1,5-pentanediol (“1,5-PD”). The 1,5-PD itself is an expensive reactant, thus yielding a very high current market price for DVL (˜$800/kg—year 2022). Because DVL is currently produced from petroleum, its market price is also directly linked to the supply, demand, and political conditions of the broader crude oil markets.

For example, a route from 1,5-PD to DVL is described in U.S. Pat. No. 8,466,299, issued Jun. 18, 2013, to Pinkos et al. In this approach, 1,5-pentanediol is evaporated into an inert gas stream and then passed over a catalyst bed. The catalyst bed includes at least two different copper-based catalysts in a layered structure so that the gas stream contacts the two different catalysts in a serial fashion—one after the other. The catalyst bed is temperature controlled to have an inlet temperature of from 260 to 350° C. and an outlet temperature of from 180 to 240° C. The pressure of the gas stream as it passes through the catalyst bed is carefully controlled so that no liquid phase is formed within the reactor itself. The gas stream exiting the reaction chamber comprises DVL, which is condensed from or otherwise separated from unreacted 1,5-PD present in the product gas stream.

Using renewable feedstocks to make DVL and other related compounds has also been explored, such as gamma-valerolactone (γ-valerolactone; “GVL”):

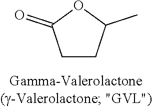

DVL can also be made from cyclopentanone using the Baeyer-Villiger reaction. See Hara, Hatakeyama, Kim, Ichikuni, and Shimazu (2012) Green Chem 14:771-777; Cavarzan, Scarso, Sgarbossa, Michelin, and Strukul (2010) ChemCatChem 2:1296-1302; and Ten Brink and Sheldon (2004) Chem. Rev. 104:4105-4123. The reaction, however, requires expensive peroxo complexes and very harsh reaction conditions. It thus is not an economically feasible route for large-scale production.

Schniepp and Geller (Aug. 1946) J. Amer. Chem. Soc. 68(8):1646-1648, describe a route to 1,5-PD in which tetrahydrofurfural alcohol (“THFA”) is converted in the presence of activated alumina to 3,4-dihydropyran (“DHP”). The DHP is then hydrated with a homogeneous acid catalyst (0.2 N HCl) to yield a mixture that was approximately 84 wt % gamma-hydroxyvaleraldehyde (i.e., 5-hydroxyvaleraldehyde, also synonymous with 5-hydroxypentanal “5-HP”) and 16 wt % 2-hydroxytetrahydropyran (“HTHP”). The reaction mixture is neutralized with base (0.4 N NaOH). Notably, the two intermediates in the mixture were separated via reduced pressure distillation. The last step is performed solely on the 5-HP, which is reduced (hydrogenated) to 1,5-PD. The overall yield of 1,5-PD from THFA, when the 5-HP and HTHP were separated prior to the final step, was 70%. However, without the intermediate isolation of the 5-HP, the overall yield of 1,5-PD dropped to 60-62%. In both instances, the DHP intermediate was subjected to acid hydrolysis with homogeneous HCl catalysts.

More recently, the literature reveals a route only partially dependent on using 1.5-pentanediol in the feedstock, along with ethyl levulinate derived from biomass. Here, GVL and DVL are produced simultaneously in the gas phase, again using copper catalysts. In this approach, dehydrogenation of 1,5-pentanediol is catalytically coupled to hydrogenation of ethyl levulinate to yield a product mixture containing both DVL and GVL. The dehydration of 1,5-pentanediol to DVL is endothermic and requires 1 mole of 1,5-pentanediol and 61.6 kJ of energy to yield 1 mole of DVL:

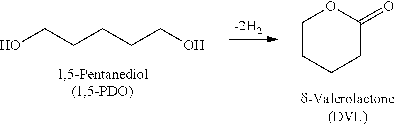 [SMILES: OCCCCCO ; O=C1CCCCO1]

This reaction is coupled to the exothermic hydrogenation of ethyl levulinate (EL) to GVL, which requires 1 mole of ethyl levulinate and 1 mole of H₂ to yield 1 mole of GVL (and 1 mole of ethanol), and releases 211 LI of energy:

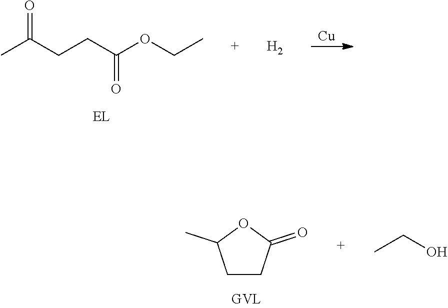

Thus, on a “per mole” basis, the hydrogenation of ethyl levulinate to GVL yields more than enough heat (produces 211 kJ per mole) to drive the dehydration of 1,5-pentanediol to DVL (requires 61.6 LI per mole). See Wang, Ding, Li, She, Zhu, and Li (2021) “Sustainable production of γ-valerolactone and δ-valerolactone through the coupling of hydrogenation and dehydrogenation,” Sustainable Energy Fuels 5:930-934. The reaction, however, still requires using petroleum-derived 1,5-pentanediol as a feedstock.

1,5-Pentanediol can be made from renewable sources. See U.S. Pat. No. 10,183,904, issued Jan. 22, 2019, to Huber, Dumesic, Barnett, and Brentzel, and U.S. Pat. No. 10,077,224, issued Sep. 18, 2018, to Huber, Dumesic, and Barnett. In this approach, tetrahydrofurfuryl alcohol (THFA) is converted to 1,5-pentanediol via a dehydration, hydration, hydrogenation (“DHH”) pathway. See FIG. 1.

Thus, there remains a long-felt and unmet need for a method to produce DVL that is sustainable (i.e., utilizes a renewable, non-petroleum-based feedstock), scalable, and economically feasible.

### SUMMARY

Disclosed herein is a method to make delta-valerolactone. The method comprises dehydrogenating 2-hydroxytetrahydropyran (“HTHP”) by contacting the HTHP with a supported-metal catalyst for a time, at a temperature, and at a pressure wherein at least a portion of the HTHP is converted to delta-valerolactone (“DVL”). The reaction feedstock can be neat or an aqueous solution having a concentration of from about 5 wt % to about 99 wt % HTHP. The method is preferably conducted in the gas phase. The reaction may also proceed in the absence of a catalyst—i.e., thermally. That is, the reaction does occur thermally, in an empty reactor tube that is heated but does not contain any catalyst. This is because the formation of DHP and DVL from the HTHP reactant is thermodynamically spontaneous (ΔG_{reaction}<0) at temperatures higher than 0° C. The thermal route, however, is not preferred because the HTHP conversion is low (<2%; data not shown) without catalyst.

The method may be conducted in the presence of an inert feed gas in order to control the flow rate of the reactants through the catalyst bed. The inert feed gas is preferably selected from the group consisting of Ar, He, and N₂.

The temperature at which the reaction is conducted can vary widely. Preferably, the HTHP is contacted with the catalyst at a temperature of from about 100° C. to about 300° C. Temperatures above and below this preferred range are explicitly included herein. Likewise, the pressure of the reaction may vary widely, preferably from ambient atmospheric pressure to about 500 psi (about 3.5 MPa). Again, pressures above and below this preferred range are explicitly included herein.

The catalyst preferably comprises (but is not limited to) a metal selected from the group consisting of Ti, V, Fe, Co, Cu, Ni, Mo, Rh, Ru, Pd, Au, Ag, Ir, Re, Pt, and combinations thereof. The catalyst is preferably disposed on a support. A host of suitable supports are known. Preferred supports include carbon and oxides of Si, Al, Mg, and Al and combinations thereof. Other suitable supports include refractory oxides such as (but not limited to) zirconia, titania, hafnia, and mixtures thereof. The catalyst support material can be or can include rare earth-modified refractory metal oxides, where the rare earth may be any rare earth metal, for example, lanthanum or yttrium; and/or alkali earth metal-modified refractory oxides. The catalyst support material can be categorized as materials having a substantially stable surface area at reaction conditions, for example, a surface area that is not substantially altered by reaction conditions or altered in a way that affects the reaction.

The HTHP reactant may be present in a feed mixture further comprising a compound selected from the group consisting of 5-hydroxypentanal, 2,2′ -hydroxytetrahydropyran, tetrahydropyran-oxypentanal, and combinations thereof. These compounds may be provided in an equilibrium mixture.

The method may yield a product mixture comprising DVL, 3,4-dihydropyran 35 (“DHP”), and 1,5-pentanediol (“1,5-PD”).

Because the method produces DVL from furfural-derived HTHP, petroleum can be replaced with a renewable biomass feedstock and the DVL production costs can be reduced to ˜$2-3/kg. Moreover, recent research suggests high molecular weight polyDVL has mechanical properties that eclipse even high-density polyethylene (HDPE). Lowering the price of DVL manufacturing will enable the production of economically competitive polyesters with enhanced properties.

The method can also be carried out at relatively mild reaction conditions, without the need of added homogenous acid (and thus without the need to neutralize any acid). The method also incurs far lower separation costs. The method results in >90% overall yields to DVL (based on HTHP).

When the HTHP is produced by hydration of DHP, it is preferable to separate the HTHP from the remaining water in the product solution for subsequent production of DVL. Thus, also disclosed herein is a method to extract HTHP from an aqueous solution, the method comprising adding tetrahydropyran (THP) to the aqueous solution to form a water phase and a THP phase, wherein the HTHP enters the THP phase; and separating the water phase from the THP phase.

The method may further comprise adding molecular sieves to the THP phase to remove remaining water in the THP phase. Non-limiting examples of the molecular sieves include hygroscopic aluminosilicate beads. Preferably, the THP is added to the aqueous solution in a mass ratio of 1:1 with HTHP, but this is not intended to be limiting. The objects and advantages of the disclosure will appear more fully from the following detailed description of the preferred embodiment of the disclosure made in conjunction with the accompanying drawings.

### BRIEF DESCRIPTION OF THE DRAWINGS

FIG. 1 depicts a method of converting tetrahydrofurfuryl alcohol into 1,5-pentanediol. See U.S. Pat. Nos. 10,183,904 and 10,077,224.

FIG. 2 depicts exemplary (non-limiting) reactions of the method disclosed and claimed herein.

FIG. 3 depicts an exemplary (non-limiting) reaction scheme to make DVL from 2-hydroxytetrahydropyran.

FIG. 4 depicts a series of exemplary (non-limiting) reactions to make various types of polyesters from DVL.

FIG. 5 is a flow chart illustrating how to make DVL according to the method disclosed and claimed herein.

FIG. 6 is a schematic diagram of an exemplary (non-limiting) continuous flow reactor that can be used to implement the method disclosed herein. “MFC”=mass flow controller. “BPR”=back-pressure regulator. “GC”=gas chromatograph.

FIGS. 7A and 7B are graphs showing the effect of temperature on the selectivity of the dehydration reaction (HTHP to DVL). Both figures show carbon balance (solid line), HTHP conversion (dashed line), DVL yield (—●—), DHP yield (—▪—), and 1,5-PD (i.e., “PDO”) yield (—▴—). FIG. 7A shows yields at 175° C. FIG. 7B shows yields at 250° C.

FIGS. 8A, 8B, and 8C are graphs showing the effect of WHSV on selectivity to DVL. The key is the same as for FIGS. 7A and 7B. FIG. 8A shows yields at WHSV=2.7 h⁻¹. FIG. 8B shows yields at WHSV=0.7 h⁻¹. FIG. 8C shows yields at WHSV=0.3 h⁻¹.

FIG. 9 is a graph showing production of DVL according to an exemplary version of the method disclosed and claimed herein. Reaction conditions: 1 atm, 0.1 mL/min HTHP in THP solution, 100 mL/min Ar, 0.1 g 20% Cu/SiO₂ catalyst, 24 h at each temperature with fresh bed of catalyst. The reaction yielded 66% selectivity to DVL at 150° C.

FIG. 10. Product rates from the thermocatalytic reactions of HTHP over various metal oxides and supported-transition metal catalysts. Reaction conditions: 150° C., 2 mM HTHP in THP, 200 psi Ar, 45 mL Parr reactor, 800 rpm, 30 minutes. With the exception of CuO/SiO₂, all other transition metal catalysts were reduced in situ before reaction. The stacked bars show production rate of humins, PDO, DHP, D2, D1, and DVL from top to bottom.

FIGS. 11A-11D. Effect of temperature on the thermal equilibrium between HTHP, DHP, D1, and D2 at: FIG. 11A) 100° C., FIG. 11B) 125° C., FIG. 11C) 135° C., and FIG. 11D) 150° C. Reaction conditions: 1 mM HTHP, 45 mL Parr reactor, 800 rpm, 200 psi Ar. Solid lines are model-predicted values.

FIGS. 12A-12D. Effect of feed composition on the thermal equilibrium between HTHP, DHP, D1 and D2 for the following feeds: FIG. 12A) 0.1 mM HTHP, FIG. 12B) 2 mM HTHP, FIG. 12C) 2 mM HTHP+1 mM DHP, and FIG. 12D) 1 mM HTHP+1 mM DHP. Reaction conditions: 125° C., 45 mL Parr reactor, 800 rpm, 200 psi Ar. Solid lines are model-predicted values.

FIGS. 13A-13B. Effect of reaction temperature on: FIG. 13A) turnover frequency (TOF), and FIG. 13B) selectivity and HTHP conversion. Reaction conditions: 125-250° C., 1.1 mM contact time, 0.1 mL/min 10 wt % HTHP in THP, 0.1 g Cu/SiO₂, 50 mL/min Ar, 200 psi, 24 h at each contact time. Clustered columns show TOF and selectivity of D1, DHP, D2, DVL, and humins from left to right.

FIGS. 14A-14B. Effect of contact time on: FIG. 14A) turnover frequency (TOF), and FIG. 14B) selectivity and HTHP conversion. Reaction conditions: 150° C., 0.5-31.5 min contact time, 10 wt % HTHP in THP, 50 mL/min Ar, 200 psi, 24 h at each contact time, Cu/SiO₂. Clustered columns show TOF and selectivity of D1, DHP, D2, DVL, and humins from left to right.

FIG. 15. Log-log plots of product turnover frequencies (TOF) versus HTHP partial pressure (P_{HTHP}for determination of reaction orders (n) from the slope of each curve.

Reaction conditions: 150° C., 1 min contact time, 0.1 mL/min HTHP in THP, 0.1 g Cu/SiO₂, 200 psi, 24 h at each partial pressure. Solid lines are visual aids.

FIGS. 16A-16B. Effect of time on stream (TOS) on: FIG. 16A) turnover frequency (TOF) and carbon balance, and FIG. 16B) product selectivity and HTHP conversion. Reaction conditions: 150° C., 0.07 min⁻¹ WHSV, 0.1 g 20wt % Cu/SiO₂, 50 mL/min Ar, 200 psi, 72 h time on stream. Solid lines are visual aids.

FIG. 17. Model-predicted selectivity to DVL from dehydrogenation of HTHP over Cu/SiO₂ as a function of temperature (125-150° C.) and contact time (0-40 minutes). DVL selectivity is maximized at 150° C. and 22 minutes.

### DETAILED DESCRIPTION

### Abbreviations and Definitions

1,5-PD=1,5-pentanediol. 5-HP=5-hydroxypentanal, which is synonymous with 5-hydroxyvaleraldehyde and gamma-hydroxyvaleraldehyde. BPR=back-pressure regulator. DHP=3,4-dihydropyran. DVL=delta-valerolactone (δ-valerolactone). EL=ethyl levulinate. GC=gas chromatograph. GVL=gamma-valerolactone (γ-valerolactone). HMF=5-hydroxymethylfurfural. HTHP=2-hydroxytetrahydropyran. MFC=mass flow controller. PVL=poly(valerolactone). THFA=tetrahydrofurfuryl alcohol. TOF=turnover frequency. TOS=time on stream. WHSV=weight hourly space velocity.

“Biomass” as used herein includes materials containing cellulose, hemicellulose, lignin, protein and carbohydrates such as starch and sugar. Common forms of biomass include trees, shrubs and grasses, corn and corn husks as well as municipal solid waste, waste paper and yard waste. Biomass high in starch, sugar or protein such as corn, grains, fruits and vegetables, is usually consumed as food. Conversely, biomass high in cellulose, hemicellulose and lignin is not readily digestible by humans and is primarily utilized for wood and paper products, fuel, or is discarded as waste. “Biomass” as used herein explicitly includes branches, bushes, canes, corn and corn husks, energy crops, forests, fruits, flowers, grains, grasses, herbaceous crops, leaves, bark, needles, logs, roots, saplings, short rotation woody crops, shrubs, switch grasses, trees, vegetables, vines, hard and soft woods. In addition, biomass includes organic waste materials generated from agricultural processes including farming and forestry activities, specifically including forestry wood waste. “Biomass” includes virgin biomass and/or non-virgin biomass such as agricultural biomass, commercial organics, construction and demolition debris, municipal solid waste, waste paper, and yard waste. Municipal solid waste generally includes garbage, trash, rubbish, refuse and offal that is normally disposed of by the occupants of residential dwelling units and by business, industrial and commercial establishments, including but not limited to: paper and cardboard, plastics, food scraps, scrap wood, saw dust, and the like.

“Biomass-derived” as used herein refers to compounds or compositions fabricated or purified from biomass. Glucose and HMF for use in the disclosed method may be biomass derived.

Broisted-Lowry Acid/Base: A Bronsted-Lowry acid is defined herein as any chemical species (atom, ion, molecule, compound, complex, etc.), without limitation, that can donate or transfer one or more protons to another chemical species. Mono-protic, diprotic, and triprotic acids are explicitly included within the definition. A Bronsted-Lowry base is defined herein as any chemical species that can accept a proton from another chemical species. Included among Bronsted-Lowry acids are mineral acids, organic acids, heteropolyacids, solid acid catalysts, zeolites, etc. as defined herein. Note that this list is exemplary, not exclusive. The shortened term “Bronsted” is also used synonymously with “Bronsted-Lowry.”

“Carbohydrate” is defined herein as a compound that consists only of carbon, hydrogen, and oxygen atoms, in any ratio.

“C₅ carbohydrate” refers to any carbohydrate, without limitation, that has five (5) carbon atoms. The definition includes pentose sugars of any description and stereoisomerism (e.g., D/L aldopentoses and D/L ketopentoses). C₅ carbohydrates include (by way of example and not limitation) arabinose, lyxose, ribose, ribulose, xylose, and xylulose. “C₆ carbohydrate” refers to any carbohydrate, without limitation, that has six (6) carbon atoms. The definition includes hexose sugars of any description and stereoisomerism (e.g., D/L aldohexoses and D/L ketohexoses). C₆ carbohydrates include (by way of example and not limitation) allose, altrose, fructose, galactose, glucose, gulose, idose, mannose, psicose, sorbose, tagatose, and talose.

“Cellulose” refers to a polysaccharide of glucose monomers ((C₆H₁₀O₅)ₙ); “cellulosic biomass” refers to biomass as described earlier that comprises cellulose, and/or consists essentially of cellulose, and/or consists entirely of cellulose. Lignocellulosic biomass refers to biomass comprising cellulose, hemicellulose, and lignin. Lignocellulosic biomass comprises xylose, as does hemicellulose.

“Dehydration catalyst” means any catalyst, without limitation, whether now known or developed in the future, capable of removing water from organic compounds.

“Glucose-containing oligomers, glucose-containing polymers, Glucose-containing reactant, C6-containing reactant”=Any chemical species, having any type of intramolecular bond type, that comprises a glucose unit. The definition explicitly includes glucose-containing disaccharides (such as, but not limited to, sucrose, lactose, maltose, trehalose, cellobiose, kojibiose, nigerose, isomaltose, β-β-trehalose, α,β-trehalose, sophorose, laminaribiose, gentiobiose, turanose, maltulose, palatinose, gentiobiulose, etc.), trisaccharides (such as, but not limited to, isomaltotriose, nigerotriose, maltotriose, maltotriulose, raffinose, etc.), and larger oligosaccharides and polysaccharides, as well as large and more complex glucose-containing polymers and carbohydrates, such as, but not limited to, starch, amylase, amylopectin, glycogen, cellulose, hemicelluloses (e.g., xyloglucan, glucomannan, etc.), lignocellulose, and the like. Linear, branched, and macrocyclic oligomers and polymers containing glucose are explicitly included within the definition.

“Heteropolyacid” as used herein refers to a class of solid-phase acids exemplified by such species as H₄ SiW₁₂O₄₀, H₃PW₁₂O₄₀, H₆P₂W₁₈O₆₂, H₃₊ₓPMo₁₂₋ₓVₓO₄₀ and the like. Heteropolyacids are solid-phase acids having a well-defined local structure, the most common of which is the tungsten-based Keggin structure. The Keggin unit comprises a central PO₄ tetrahedron, surrounded by 12 WO₆ octahedra. The standard unit has a net (⁻3) charge, and thus requires three cations to satisfy electroneutrality. If the cations are protons, the material functions as a Bronsted acid. The acidity of these compounds (as well as other physical characteristics) can be “tuned” by substituting different metals in place of tungsten in the Keggin structure. See, for example, Bardin et al. (1998) “Acidity of Keggin-Type Heteropolycompounds Evaluated by Catalytic Probe Reactions, Sorption Microcalorimetry and Density Functional Quantum Chemical Calculations,” J. of Physical Chemistry B, 102:10817-10825.

“Homogeneous catalyst” as used herein refers to a catalyst that exists in the same phase (solid, liquid, or gas) as the reactants under reaction conditions. “Heterogeneous catalyst” as used herein refers to a catalyst that exists in a different phase than the reactants under reaction conditions.

“Hydrofuran” is used herein to refer to any unsubstituted or substituted cyclic ether having a single oxygen heteroatom in the ring, and having five total atoms in the ring and which is derived from furanic compounds. Hydrofurans that are miscible in water, such as tetrahydrofuran (THF), are more appropriate for use in the monophasic reactions described herein. In the biphasic reactions, any hydrofuran may be used.

“Hydropyran” is used herein to refer to any unsubstituted or substituted cyclic ether having a single oxygen heteroatom in the ring, and having six total atoms in the ring and which is derived from pyranic compounds. Hydropyrans miscible in water are more appropriate for use in the monophasic reactions described herein. In the biphasic reactions, any hydropyran may be used.

Mineral acid as used herein refers to any mineral-containing acid, including (by way of example and not limitation), hydrochloric acid, nitric acid, phosphoric acid, SA, boric acid, hydrofluoric acid, hydrobromic acid, and the like.

Lewis Acid/Base: Lewis acid is defined herein as any chemical species that is an electron-pair acceptor, i.e., any chemical species that is capable of receiving an electron pair, without limitation. A Lewis base is defined herein as any chemical species that is an electron-pair donor, that is, any chemical species that is capable of donating an electron pair, without limitation.

The Lewis acid (also referred to as the Lewis acid catalyst) may be any Lewis acid based on transition metals, lathanoid metals, and metals from Group 4, 5, 13, 14 and 15 of the periodic table of the elements, including boron, aluminum, gallium, indium, titanium, zirconium, tin, vanadium, arsenic, antimony, bismuth, lanthanum, dysprosium, and ytterbium. One skilled in the art will recognize that some elements are better suited in the practice of the method. Illustrative examples include AlCl₃, (alkyl)AlCl₂, (C₂H₅)₂AlCl, (C₂H₅)₃Al₂Cl₃, BF₃, SnCl₄ and TiCl₄.

The Group 4, 5 and 14 Lewis acids generally are designated by the formula MX4; wherein M is Group 4, 5, or 14 metal, and X is a halogen independently selected from the group consisting of fluorine, chlorine, bromine, and iodine, preferably chlorine. X may also be a psuedohalogen. Non-limiting examples include titanium tetrachloride, titanium tetrabromide, vanadium tetrachloride, tin tetrachloride and zirconium tetrachloride. The

Group 4, 5, or 14 Lewis acids may also contain more than one type of halogen. Non-limiting examples include titanium bromide trichloride, titanium dibromide dichloride, vanadium bromide trichloride, and tin chloride trifluoride.

Group 4, 5 and 14 Lewis acids useful in the method may also have the general formula MRₙX₄₋ₙ; wherein M is Group 4, 5, or 14 metal; wherein R is a monovalent hydrocarbon radical selected from the group consisting of C₁ to C₁₂ alkyl, aryl, arylalkyl, alkylaryl and cycloalkyl radicals; wherein n is an integer from 0 to 4; and wherein X is a halogen independently selected from the group consisting of fluorine, chlorine, bromine, and iodine, preferably chlorine. X may also be a psuedohalogen. Non-limiting examples include benzyltitanium trichloride, dibenzyltitanium dichloride, benzylzirconium trichloride, dibenzylzirconium dibromide, methyltitanium trichloride, dimethyltitanium difluoride, dimethyltin dichloride and phenylvanadium trichloride.

Group 4, 5 and 14 Lewis acids useful in method may also have the general formula M(RO)ₙR′ₘX₍ₘ₊ₙ₎; wherein M is Group 4, 5, or 14 metal; RO is a monovalent hydrocarboxy radical selected from the group consisting of C₁ to C₃₀ alkoxy, aryloxy, arylalkoxy, alkylaryloxy radicals; R′ is a monovalent hydrocarbon radical selected from the group consisting of C₁ to C₁₂ alkyl, aryl, arylalkyl, alkylaryl and cycloalkyl radicals; n is an integer from 0 to 4; m is an integer from 0 to 4 such that the sum of n and m is not more than 4; and X is a halogen independently selected from the group consisting of fluorine, chlorine, bromine, and iodine, preferably chlorine. X may also be a psuedohalogen. Non-limiting examples include methoxytitanium trichloride, n-butoxytitanium trichloride, di(isopropoxy)titanium dichloride, phenoxytitanium tribromide, phenylmethoxyzirconium trifluoride, methyl methoxytitanium dichloride, methyl methoxytin dichloride and benzyl isopropoxyvanadium dichloride.

Group 5 Lewis acids may also have the general formula MOX₃; wherein M is a Group 5 metal; X is a halogen independently selected from the group consisting of fluorine, chlorine, bromine, and iodine, preferably chlorine. A non-limiting example is vanadium oxytrichloride.

The Group 13 Lewis acids have the general formula MX₃; wherein M is a Group 13 metal and X is a halogen independently selected from the group consisting of fluorine, chlorine, bromine, and iodine, preferably chlorine. X may also be a psuedohalogen. Non-limiting examples include aluminum trichloride, boron trifluoride, gallium trichloride, indium trifluoride, and the like.

The Group 13 Lewis acids useful in method may also have the general formula: MRₙX₃₋ₙ wherein M is a Group 13 metal; R is a monovalent hydrocarbon radical selected from the group consisting of C₁ to C₁₂ alkyl, aryl, arylalkyl, alkylaryl and cycloalkyl radicals; and n is an number from 0 to 3; and X is a halogen independently selected from the group consisting of fluorine, chlorine, bromine, and iodine, preferably chlorine. X may also be a psuedohalogen. Non-limiting examples include ethylaluminum dichloride, methylaluminum dichloride, benzylaluminum dichloride, isobutylgallium dichloride, diethylaluminum chloride, dimethylaluminum chloride, ethylaluminum sesquichloride, methylaluminum sesquichloride, trimethylaluminum and triethylaluminum.

Group 13 Lewis acids useful in this disclosure may also have the general formula M(RO)ₙR′ₘX₃₋₍ₘ₊ₙ₎; wherein M is a Group 13 metal; RO is a monovalent hydrocarboxy radical selected from the group consisting of C₁ to C30 alkoxy, aryloxy, arylalkoxy, alkylaryloxy radicals; R′ is a monovalent hydrocarbon radical selected from the group consisting of C₁ to C₁₂ alkyl, aryl, arylalkyl, alkylaryl and cycloalkyl radicals; n is a number from 0 to 3; m is an number from 0 to 3 such that the sum of n and m is not more than 3; and X is a halogen independently selected from the group consisting of fluorine, chlorine, bromine, and iodine, preferably chlorine. X may also be a psuedohalogen. Non-limiting examples include methoxy aluminum dichloride, ethoxy aluminum dichloride, 2,6-di-tert-butylphenoxyaluminum dichloride, methoxy methylaluminum chloride, 2,6-di-tert-butylphenoxy methylaluminum chloride, isopropoxygallium dichloride and phenoxy methylindium fluoride.

Group 13 Lewis acids useful in this disclosure may also have the general formula M(RC(O)O)ₙR′ₘX₃₋₍ₘ₊ₙ₎; wherein M is a Group 13 metal; RC(O)O is a monovalent hydrocarbacyl radical selected from the group consisting of C₂ to C₃₀ alkacyloxy, arylacyloxy, arylalkylacyloxy, alkylarylacyloxy radicals; R′ is a monovalent hydrocarbon radical selected from the group consisting of C₁ to C₁₂ alkyl, aryl, arylalkyl, alkylaryl and cycloalkyl radicals; n is a number from 0 to 3 and m is a number from 0 to 3 such that the sum of n and m is not more than 3; and X is a halogen independently selected from the group consisting of fluorine, chlorine, bromine, and iodine, preferably chlorine. X may also be a psuedohalogen. Non-limiting examples include acetoxyaluminum dichloride, benzoyloxyaluminum dibromide, benzoyloxygallium difluoride, methyl acetoxyaluminum chloride, and isopropoyloxyindium trichloride.

The most preferred Lewis acids for use in the method are metal halides generally and more specifically transition metal halides, lathanoid metal halides, and Group 5, 13, and 14 metal halides. Preferred among the metal halides are metal chlorides. Preferred transition metal chlorides include, but are not limited to, TiCl₄, VCl₃.and the like. Preferred Group 13 and 14 metal halides and chlorides include, but are not limited to, BF₃, AlCl₃, SnCl₄, InCl₃, and GaCl₃. Preferred lanthanoid chlorides include, but are not limited to, LaCl₃, DyCl₃ and YbCl₃.

“Noble metal” is used herein to include ruthenium, rhodium, palladium, silver, osmium, iridium, platinum, gold, and rhenium. Other corrosion-resistant metals that can be used as catalysts in the subject process include titanium, niobium, and tantalum.

The terms “solid acid” and “solid acid catalyst” are used synonymously herein and can comprise one or more solid acid materials. The solid acid catalyst can be used independently or alternatively can be utilized in combination with one or more mineral acid or other types of catalysts. Exemplary solid acid catalysts which can be utilized include, but are not limited to, heteropolyacids, acid resin-type catalysts, mesoporous silicas, acid clays, sulfated zirconia, molecular sieve materials, zeolites, and acidic material on a thermo-stable support. Where an acidic material is provided on a thermo-stable support, the thermo-stable support can include for example, one or more of carbon, alpha-alumina, and the like. The oxides themselves (e.g., ZrO₂, SnO₂, TiO₂, etc.) which may optionally be doped with additional acid groups such as SO₄²⁻ or SO₃H may also be used as solid acid catalysts.

Further examples of solid acid catalysts include strongly acidic ion exchangers such as cross-linked polystyrene containing sulfonic acid groups. For example, the Amberlyst®-brand resins are functionalized styrene-divinylbenzene copolymers with different surface properties and porosities. (These types of resins are designated herein as “Amb” resins, followed by a numeric identifier of the specific sub-type of resin where appropriate.) The functional group is generally of the sulfonic acid type. The Amberlyst®-brand resins are supplied as gellular or macro-reticular spherical beads. (Amberlyst® is a registered trademark of the Dow Chemical Co.) Similarly, Nafion®-brand resins are sulfonated tetrafluoroethylene-based fluoropolymer-copolymers which are solid acid catalysts. Nation® is a registered trademark of E. I. du Pont de Nemours & Co.)

Solid catalysts can be in any shape or form now known or developed in the future, such as, but not limited to, granules, powder, beads, pills, pellets, flakes, cylinders, spheres, or other shapes.

Supports for metal catalysts can be any suitable support (now known or developed in the future) that is sufficiently robust to withstand the reaction conditions disclosed herein. Suitable catalyst supports include, by way of example and not limitation, alumina, carbon, ceria, magnesia, niobia, silica, titania, zirconia, zeolites (preferably, Y, ZSM 5, MWW and beta), hydrotalcite, molecular sieves, clays, iron oxide, silicon carbide, aluminosilicates, and modifications, mixtures or combinations thereof.

Zeolites may also be used as solid acid catalysts. Of these, H-type zeolites are generally preferred, for example zeolites in the mordenite group or fine-pored zeolites such as zeolites X, Y and L, e.g., mordenite, erionite, chabazite, or faujasite. Also suitable are ultrastable zeolites in the faujasite group which have been dealuminated.

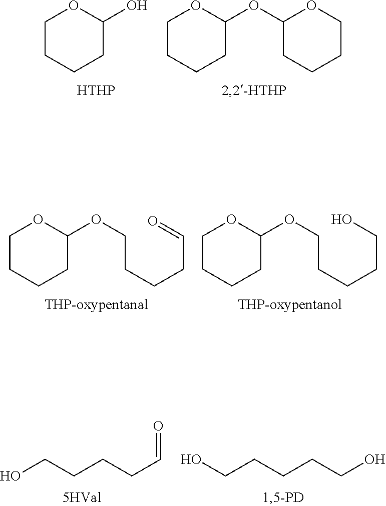

All references to singular characteristics or limitations of the disclosed method shall include the corresponding plural characteristic or limitation, and vice-versa, unless otherwise specified or clearly implied to the contrary by the context in which the reference is made. The indefinite articles “a” and “an” mean “one or more.”

All combinations of method steps disclosed herein can be performed in any order, unless otherwise specified or clearly implied to the contrary by the context in which the referenced combination is made.

The method disclosed herein can comprise, consist of, or consist essentially of the essential elements and steps described herein, as well as any additional or optional ingredients, components, or limitations described herein or otherwise useful in organic chemistry. The disclosure provided herein may be practiced in the absence of any element or step which is not specifically disclosed herein.

### The Method

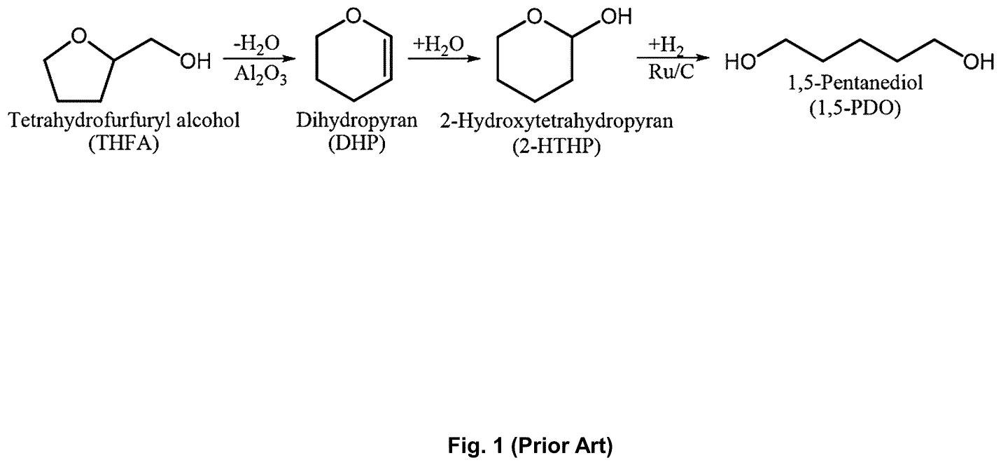

Referring now to FIG. 1, 1,5-PD (“1,5-PDO” in the figure) can be made from renewable sources such as biomass-derived THFA. See U.S. Pat. Nos. 10,183,904 and 10,077,224. The method proceeds in three steps: (1) dehydrating tetrahydrofurfural alcohol (THFA) to 3,4-dihydropyran (DHP), preferably over a γ-Al₂O₃ catalyst which afforded >93% yield to DHP; (2) hydrating at least a portion of the DHP to 2-hydroxy-tetrahydropyran (HTHP), which afforded approximately 94% yield to HTHP; and then (3) hydrogenating at least a portion of the HTHP to 1,5-pentanediol, preferably in the presence of a metal-containing catalyst.

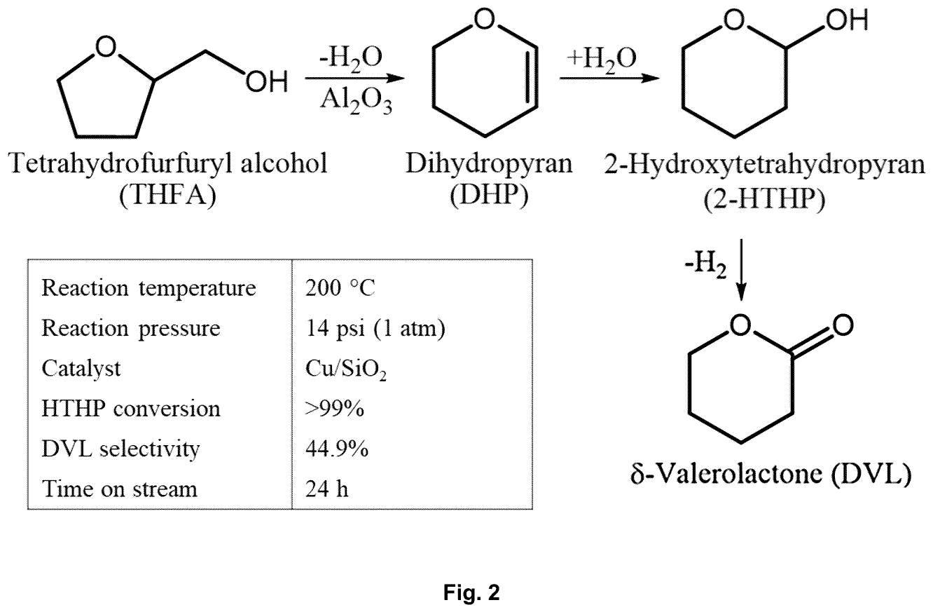

The present method may utilize the first two steps of the reaction shown in FIG. 1, to arrive at HTHP. The present method, however, then takes a different turn, as shown in FIG. 2. In the final step of the present method, rather than hydrogenating the HTHP to 1,5-PD, the HTHP is dehydrogenated to DVL. As shown in FIG. 2, under the conditions stated (200° C. reaction temperature, 14 psi (atmospheric), over a copper catalyst on a SiO₂ support, HTHP conversion was essentially quantitative (>99%) with a 44.9% selectively to the desired DVL product.

The method disclosed herein can be conducted batch-wise, continuously, or semi-continuously.

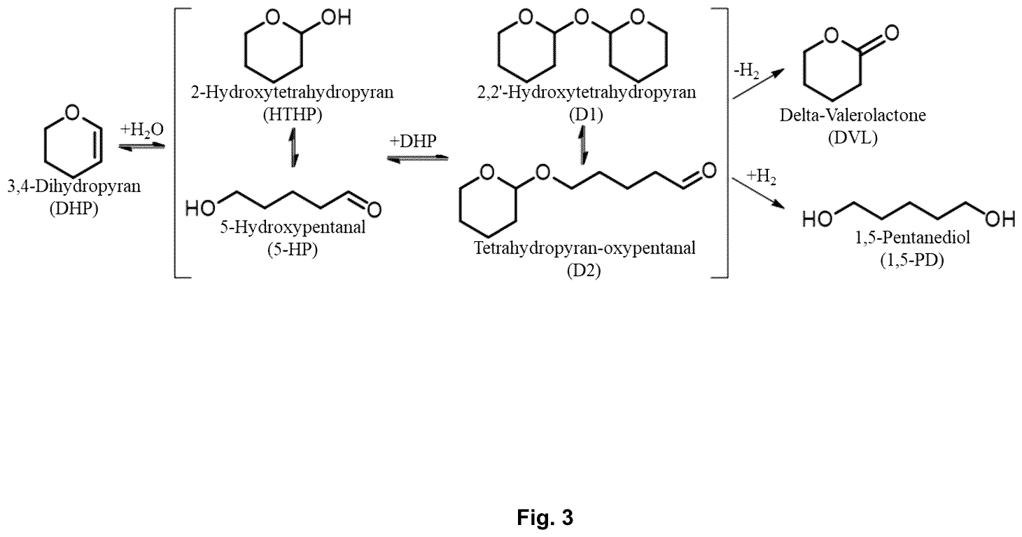

Without being limited to any specific underlying phenomenon, FIG. 3 illustrates the proposed mechanisms that account for the selectivity of the dehydrogenation reaction from 2HTHP to DVL. The hydration reaction from DHP to HTHP produces an aqueous HTHP feed containing an equilibrium mixture HTHP, 5-hydroxypentanal (5-HP), and two dimers—2,2′-hydroxytetrahydropyran (“D1” in FIG. 3) and tetrahydropyran-oxypentanal (“D2” in FIG. 3). The dehydrogenation of HTHP to the desired DVL product (upper-right in FIG. 3) occurs simultaneously with an undesired hydrogenation of the HTHP to 1,5-PD (with the hydrogen liberated by the dehydrogenation of HTHP to DVL helping to drive a simultaneous hydrogenation of HTHP to 1,5-PD).

The present method does not require noble metal catalysts. The method, however, may utilize noble metal catalysts. It also operates at higher reactant concentrations, so energy costs relating to product distillation are slashed as well. DVL can be produced via the present method for an estimated cost of approximately $77/ton (2022).

Table 1 shows the results for continuous flow reactions after 12 hours time on stream when using various solid acid catalysts for the first step, dehydration of THFA to DHP:

TABLE 1
Vapor-Phase THFA Dehydration: Acid Catalysts

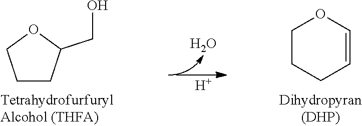

| | Mass of | Rxn | | | | | Selectivity to | Selectivity to |
|---|---|---|---|---|---|---|---|---|
| | Catalyst | Temp | Pressure | Reactant | Solvent | Conversion | DHP | Unknown |
| Catalyst | (mg) | (° C.) | (atm) | (wt %) | (wt %) | (%) | (%) | Products (%) |
| γ-Al₂O₃ | 150 | 375 | 1 | THFA-100% | N/A | 100 | 93 | 0.5 |
| Silica Alumina | 25 | 350 | 1 | THFA-20% | MeOH-80% | 95 | 46 | 10 |
| ZrO₂ | 130 | 375 | 1 | THFA-100% | N/A | 40 | 80 | 6 |

Table 2 depicts the results of the hydration reaction, DHP to HTHP:

TABLE 2
DHP to HTHP

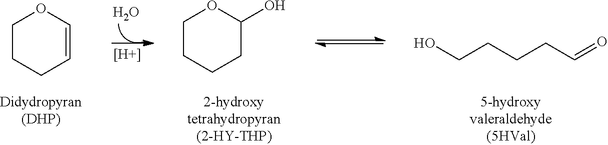

| | Reaction | Reaction | Reactant | Solvent | Conversion | Selectivity to |
|---|---|---|---|---|---|---|
| Catalyst | Temp (° C.) | Time (hr) | (wt %) | (wt %) | (%) | HTHP (%) |
| N/A | 70 | 12 | DHP-50% | H₂O-50% | 100 | 94 |

As shown in Table 2, the DHP can be converted into HTHP in very high yields (94%). This step represents a departure from the Schniepp and Geller (1946) approach, which seeks to maximize the production of 5-HP, rather than HTHP as in the present method (94%; see Table 2). Schniepp and Geller fail to recognize that the HTHP can be maximized more than the yield to 5-HP. Because the final dehydrogenation step to DVL is substantially quantitative, the overall yield of the present method (to DVL) is much improved as compared to the Schniepp and Geller route (to 1,5-PD).

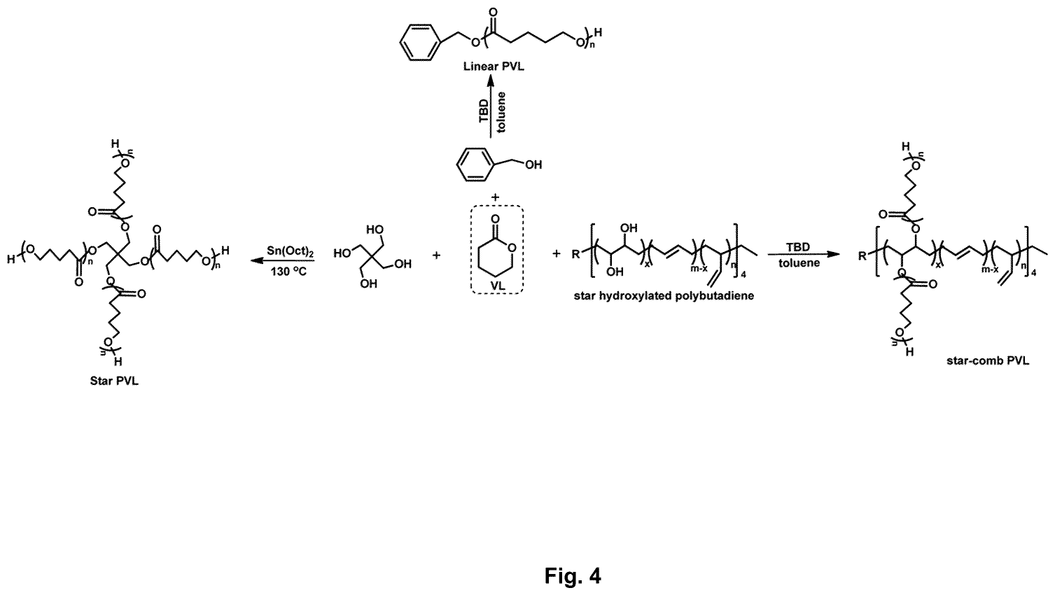

With the DVL in hand, the DVL may be polymerized in a host of different ways, examples of which are shown in FIG. 4. DVL is shown in the center. Moving upward in the figure, the DVL may be polymerized via conventional ring-opening polymerization to yield linear poly(valerolactone) (“PVL”). Moving to the left from the central DVL molecule in FIG. 4, the DVL may be co-polymerized with pentaerythritol (i.e., 2,2-bis(hydroxymethyl)propane-1,3-diol; CAS No. CAS: 115-77-5) to yield star-PVL. Moving to the right from the central DVL molecule in FIG. 4, the DVL may be co-polymerized with star-hydroxylated polybutadiene to yield star-shaped comb-type PVL.

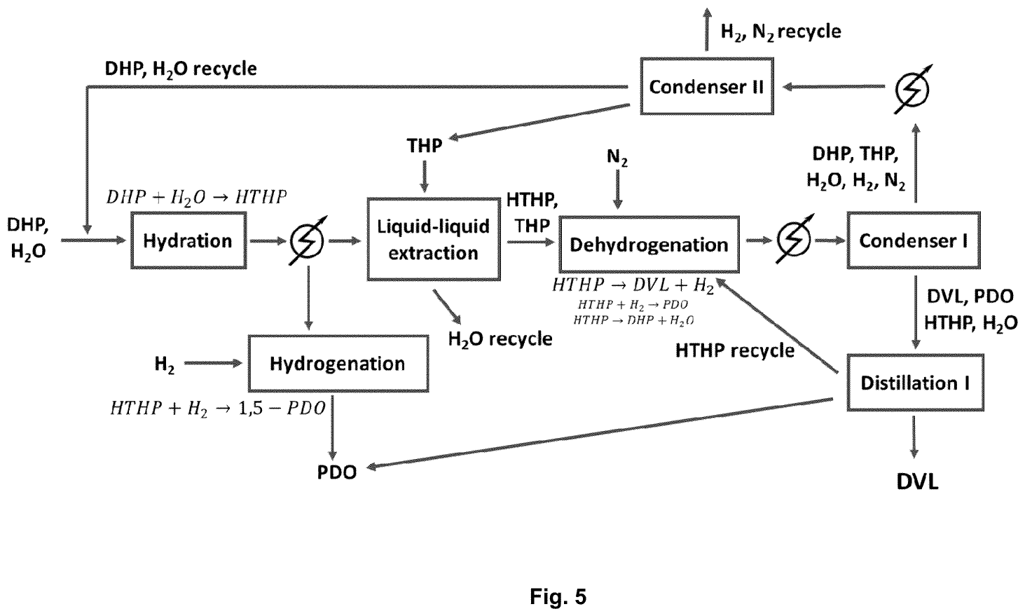

FIG. 5 is a flow chart that depicts one possible implementation of the method that accounts for the production and separation of DVL from the product mix. Moving from left-to-right across the figure, an aqueous solution of biomass-derived DHP is first subjected to hydration as described above to yield an intermediate mixture comprising HTHP. Preferably, the HTHP intermediate is further processed without any additional processing. The HTHP mixture is then subjected to dehydrogenation, as shown in the center of FIG. 5. This yields a raw product mixture comprising DVL, 1,5-PD, and any unreacted DHP and/or HTHP.

The gaseous product mixture is then condensed under controlled conditions to separate a first mixture comprising DHP, water, hydrogen, and nitrogen from a second mixture comprising DVL, 1,5-PD (“PDO” in FIG. 5), unreacted HTHP, and water. The DVL and/or 1,5-PD are then enriched, purified and/or separated from the second mixture (and from each other) by any means now known or developed in the future. In FIG. 5, the enrichment is shown as being accomplished by distillation.

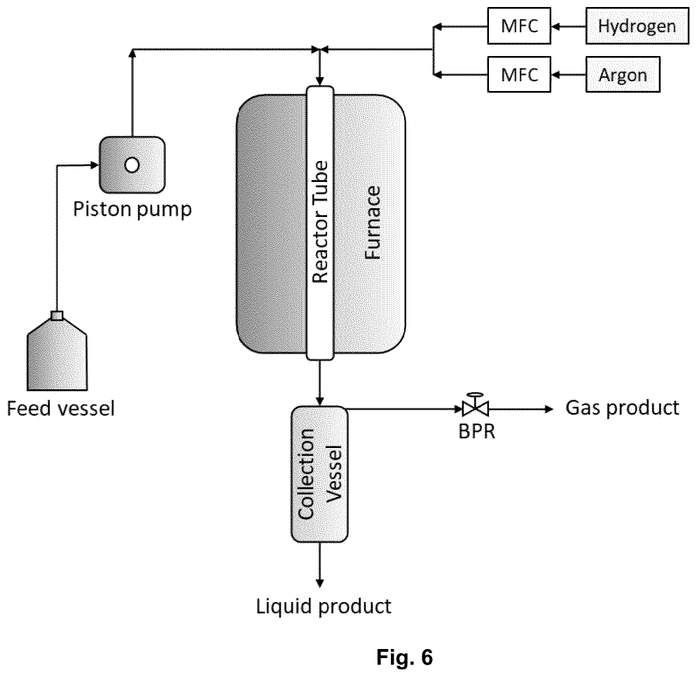

FIG. 6 is a schematic diagram of an exemplary (non-limiting) continuous flow reactor that can be used to implement the method disclosed herein. In FIG. 6, “MFC”=mass flow controller. “BPR”=back-pressure regulator. “GC”=gas chromatograph. The apparatus is designed to dehydrogenate HTHP by contacting the HTHP with a supported-metal catalyst. As shown in the figure, the catalyst is placed within a reactor tube, which is placed within a temperature-controlled furnace. (The temperature of the reactor furnace is controlled by a conventional thermostat, not shown.) The feedstock containing HTHP is introduced into the furnace from a feed vessel shown on the left of FIG. 6. After the reactor tube is placed in the furnace, hydrogen is first fed through a first MFC for several hours to reduce the catalyst in situ. Once the catalyst has been reduced, the furnace is set to the desired reaction temperature and argon is flowed through a second MFC to maintain the desired reaction pressure. The HTHP feed pump is then started for the course of the dehydrogenation reaction. The product mixture exiting the reactor tube is then collected in the product collection vessel. Gaseous products are vented (principally H₂ and Ar). The raw liquid product mix (which comprises DVL) is collected from a drain at the bottom of the product collection vessel. An optional flowmeter and in-line GC may be included to monitor the operation of the reactor system.

More specifically, a two feet-long (60.96 cm) reactor tube (316 stainless steel, ¼ inch (6.35 mm) O. D., 0.18 inch (4.572 mm) I. D.) was filled with the catalyst beds or inert glass beads (Sigma Aldrich, St. Louis, MO unwashed, 30-40 US sieve), held at each end by quartz wool. The reactor tube was contained inside a tube furnace equipped with aluminum filler rods for uniform heat distribution (not shown). Both ends of the furnace were sealed with quartz wool. The reactor was pressurized with Ar gas to the desired pressure and the back-pressure regulator at the reactor system outlet was set to the corresponding pressure to hold at pressure while allowing gas flow through the system. The reactor furnace controller was set to the desired reaction temperature. For the reaction of DHP, two separate liquid HPLC pumps (Varian ProStar 210 Series) (Agilent, Inc. Santa Clara, CA; not shown) were used due to the immiscibility of DHP in water. DHP and deionized water were flowed separately at a 1:4 mass ratio (20-50 wt % DHP) into a ¼ inch (6.35 mm) tee junction where they mixed. The DHP:H₂O mixture flowed upwards where it was combined with Ar flow at 40 mL/min sccm. Inert Ar was flowed for gas phase analysis; an experiment with no Ar flow confirmed that the gas flow did not have any noticeable effects on the DHP hydration rate. Liquid products accumulated in a stainless-steel gas—liquid separator (300 mL) at the top of the reactor. Liquid samples were collected by first closing the inlet and outlet ball valves of the reactor to isolate the system. The outlet needle valve at the bottom of the liquid collection vessel was opened to collect liquid product into a container for filtration and analysis. Inlet and outlet ball valves were re-opened to continue flow through the reactor.

The initial time-on-stream (TOS=0) is defined as the first steady state data point for activation rate experiments. This was normally taken after 8-14 h of operation. Product carbon yields were calculated on a total mol C basis as shown in Equation 1. The product concentration (mol/L) was measured by gas chromatography. Reaction rates were calculated according to Equation 2. The reactor void volume was measured to be 3.3 cm³ [void fraction(ϵ)=0.40].

$$Yield ( C \% ) = \frac{mol C product}{mol C DHP fed} = \frac{{( \frac{mol product}{L} )}_{A} \cdot {Volume}_{A} ( L ) \cdot ( \frac{mol C product}{mol product} )}{DHP flowrate ( \frac{mol C}{min} ) \cdot Total sample time ( min )} \qquad ( 1 )$$

$$Reaction Rate ( \frac{μ mol}{min \cdot L} ) = \frac{{( \frac{mol 2 HYTHP product}{L} )}_{A} \cdot {Volume}_{A} ( L ) \cdot ( \frac{1 E 6 μ mol}{mol} )}{Total sample time ( min ) \cdot Void Volume ( L )} \qquad ( 2 )$$

Gas Chromatography (GC): Reaction product solutions were placed in a separation funnel for 10 minutes. The volumes of the organic and aqueous phases were recorded by funneling each phase into a graduated cylinder. The aqueous and organic products were filtered with 0.22 μm polyethersulfone and polytetrafluoroethelyne filter membranes, respectively, before analysis. Liquid products were injected into a Shimadzu Gas Chromatograph with a Flame Ionization Detector (FID). The injection port and FID temperature was 240° C. The injection volume was 1 μL and a split ratio of 100 was used. The GC column was a Restek RTX-VMS capillary column (length: 30 m, ID: 0.25 mm, film thickness: 1.4 μm). The column temperature was held at 40° C. for 1 min, ramped at 20° C./min to 240° C., and held at 240° C. for 13 min

Gas Chromatography-Mass Spectrometry (GC-MS): Mass spectrometry of product solutions was performed on a two-dimensional (2D) gas chromatography—mass spectroscopy (GC×GC−MS) with both a FID (Agilent, 7890B) and a mass selective detector (MSD; Agilent, 5977A). A flow modulator (CFP; Agilent, G3487A) was installed to make a GC×GC system. Two capillary columns, DB-17 (Agilent, Catalog No. 121-1723) and CP-Sil 5 CB (Agilent,

Catalog No. CP7700), were set up in series with the CFP for 2D separation. H₂ carrier gas was flowed at 0.7 mL/min and 25 mL/min through the first and second dimension columns, respectively. In all experiments, both the first and second dimensions were operated in constant flow mode. Ion fragment patterns were compared to known patterns in the NIST mass spectrometry database for product identification.

NMR: Products were analyzed by proton and ¹³C NMR. A Bruker standard pulse sequence “zgig30” was used for the quantitative ¹³C experiments with the following parameters: an inter-scan relaxation delay of 12 s, a sweep width of 240 ppm centered at 110 ppm, acquiring 59,520 data points with an acquisition time of 1 s, and 128 scans. (Bruker Corp., Billerica, MA.) The ¹³C Dept-135 experiments used the Bruker standard pulse sequence “deptsp135” with the following parameters: an inter-scan relaxation delay of 2 s, a sweep width of 240 ppm centered at 110 ppm, acquiring 59,520 data points with an acquisition time of 1 s, and 128 scans. Mestrelab Research's MestReNova software was used to process the spectra and the spectra were referenced to a TMS internal standard at 0 ppm. (Mestrelab Research L. C., Santiago de Compostela, Spain.)

To establish a baseline reaction rate of thermal conversion, DHP was hydrated to HTHP using water at various temperatures in batch reactors, but no catalysts of any sort. Due to its limited solubility in water (<1 wt %), DHP forms an insoluble layer on top of water. The products (primarily HTHP) formed in the reaction enter the aqueous phase until there is a single aqueous phase at total DHP conversion. The results are tabulated in Table 3.

TABLE 3
DHP Hydration: Temperature Effects (DHP hydrated without catalyst at increasing temperatures).

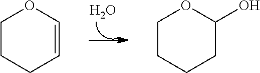

| | | | | | | |
|---|---|---|---|---|---|---|
| Temperature (° C.) | 60 | 60 | 100 | 140 | 180 | 200 |
| Reaction time (h) | 2 | 12 | 2 | 2 | 2 | 2 |
| 2-HY-THP | 21.1% | 90.6% | 92.2% | 84.3% | 59.5% | 14.8% |
| 2,2′-HY-THP | 0.3% | 2.5% | 2.3% | 1.1% | 0.6% | 0.1% |
| THP-oxypentanal | 0.4% | 5.2% | 3.8% | 5.2% | 1.9% | 0.3% |
| Total 1,5-PD Precursors | 21.8% | 98.3% | 98.3% | 90.6% | 62.0% | 15.1% |
| High T Dimer | 0.0% | 0.0% | 0.0% | 1.8% | 7.0% | 1.8% |
| Unidentified GC products | 2.6% | 1.7% | 1.6% | 2.0% | 1.8% | 2.2% |
| Missing Carbon | — | 0.0% | 0.1% | 5.7% | 29.2% | 80.8% |

Key:

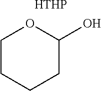

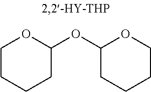 [SMILES: C1CCC(OC2CCCCO2)OC1]

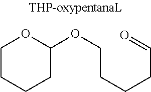 [SMILES: O=CCCCCOC1CCCCO1]

All reactions (batch and continuous) gave a final pH of about 3.4. This indicated the presence of carboxylic acids. Active acidic coke forms at the high temperatures, along with a concomitant increase in hydration rates. As can be seen from Table 3, the reactions run at 60° C. and 100° C. both yielded 98.3 wt % 1,5-PD precursors.

The hydration reaction was also run in the presence of a series of solid acid catalysts. See Table 4. These experiments revealed that a solid acid catalyst greatly increases the rate of the hydrolysis reaction. As can be seen from Table 4, the solid acid catalysts used resulted in a much-improved yield of HTHP from DHP.

TABLE 4
Comparison of HTHP production rate and Brϕnsted acid site TOF for
the hydration of 0.5 wt % DHP in DI water over various solid acid catalysts
in a batch reactor (T: 50° C., P: 500 psi Ar, Reaction time: 1 h)

| | Catalyst | HTHP | HTHP | Brϕnsted | Brϕnsted | |
|---|---|---|---|---|---|---|
| | mass | Yield | Production Rate | Site | Site Density | TOF |
| Catalyst | (mg) | (C %) | (μmol/gcat-min) | Fraction | (mmol/g) | (s⁻¹) |
| No catalyst | — | 0 | 0 | — | — | — |
| γ-Al₂O₃ | 500 | 3.8 | 4 | 0.40^{a} | 0.17 | 0.0004 |
| SiO₂—Al₂O₃ | 300 | 7.8 | 13 | 0.79^{a} | 0.72 | 0.0003 |
| CsPTA | 20 | 28.0 | 722 | — | 0.15 | 0.08 |
| ZrP | 5 | 21.1 | 2170 | 0.38^{b} | 1.05 | 0.034 |
| Nafion SAC-13 | 2 | 10.6 | 2727 | ∞^{a} | 0.14 | 0.325 |
| HZSM5 | 4 | 21.9 | 2822 | 0.51^{c} | 0.41 | 0.223 |
| H-Beta | 2 | 20.1 | 5178 | — | 2.5 | 0.035 |
| Amberlyst-15 | 0.5 | 22.0 | 22662 | ∞^{a} | 2.86 | 0.132 |
| Amberlyst-70 | 0.5 | 25.3 | 26017 | ∞^{a} | 2.86* | 0.152 |

^{a}Ronen et. al (2011);
^{b}Ronen et. al. (2013);
^{c}Yu-Ting et. al. (2012);
*Amberlyst-70 acid site density

In short, the product mix resulting from the hydration of DHP can be subjected to subsequent reactions without any isolation of the intermediate products.

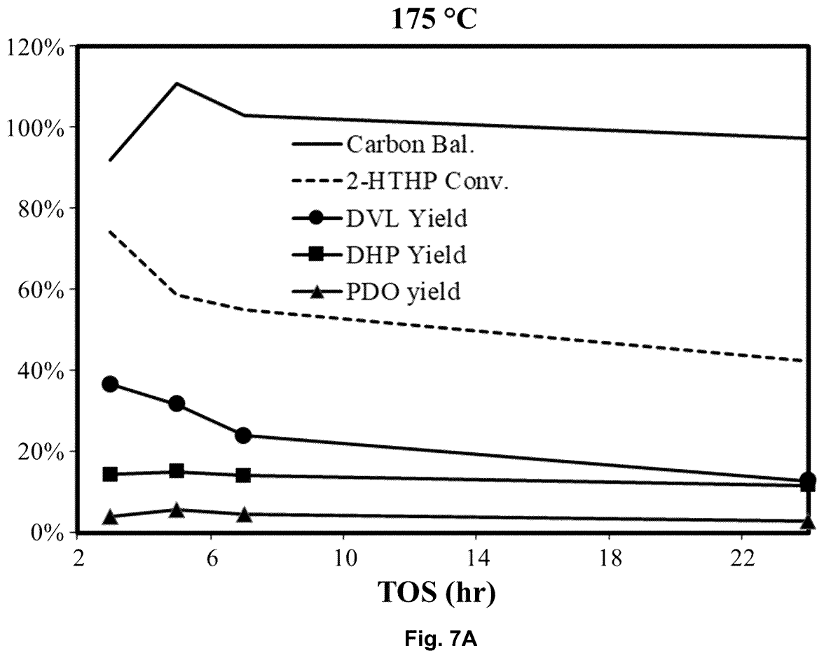

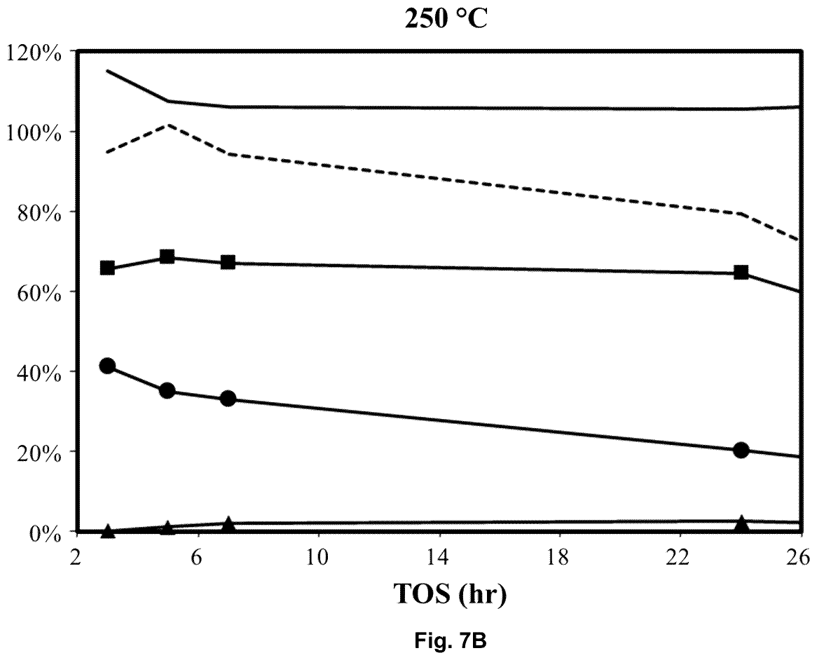

Using the reactor set up shown in FIG. 6, experiments were run under various temperatures and conditions to convert HTHP to DVL. One such set of experiments was conducted at 1 atm pressure, using lg of 10% Cu/SiO₂ supported catalyst with the reactor tube, 0.013 mL/min feed of neat 1-HTHP feed, and 100 mL/min Ar. Reactions were run at 175° C. and 250° C. The results are presented in FIGS. 7A and 7B, where carbon balance is shown by a solid line), HTHP conversion by a dashed line, DVL yield (—●—), DHP yield (—▪—), and 1,5-PD (i.e., “PDO”) yield (—▴—). FIG. 7A shows yields at 175° C. FIG. 7B shows yields at 250° C.

As can be seen from the two figures, selectivity to DVL was very similar at both temperatures. But at 250° C., selectivity to DHP was very significantly increased (all other variables remaining the same). Conversion of HTHP to product was also significantly increased at the higher temperature. These two graphs taken together show that the relative concentrations of the compounds in the product mix can be tuned using the temperature at which the reaction is conducted.

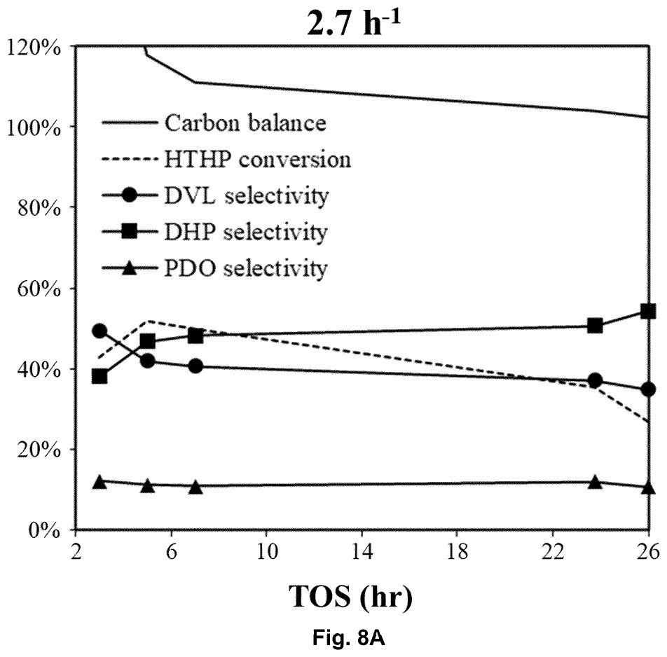

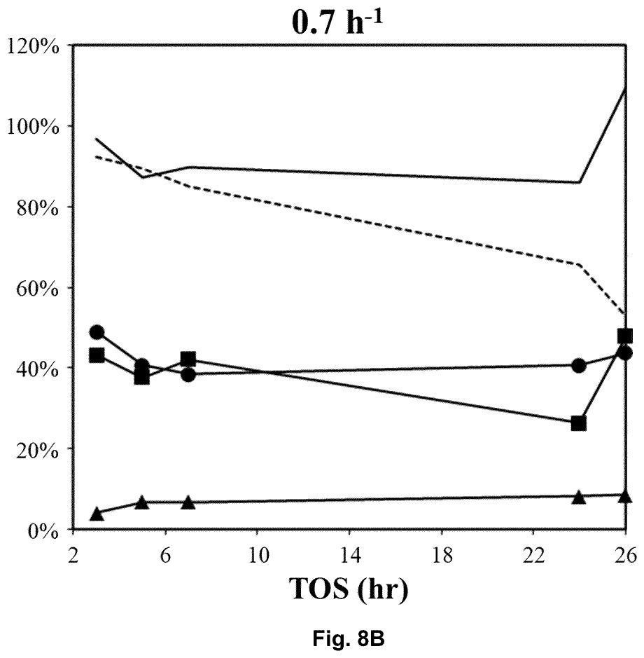

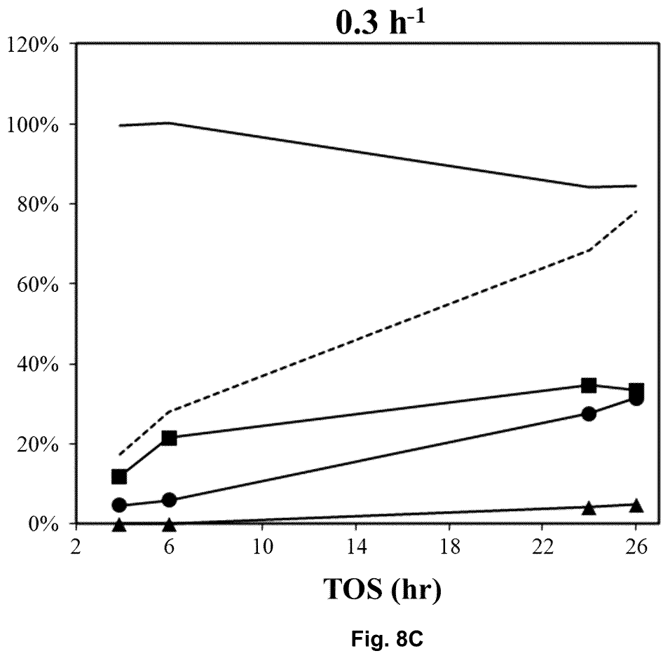

A similar set of experiments were conducted to evaluate the effect of WHSV on the selectivity of the reaction from HTHP to DVL. The results are shown in FIGS. 8A, 8B, and 8C. Here, the reactions were conducted at 200° C., 1 atm, 10% Cu/SiO₂, 0.013 mL/min feed of neat 1-HTHP feed, and 100 mL/min Ar. FIG. 8A shows yields at WHSV=2.7 h⁻¹. FIG. 8B shows yields at WHSV=0.7 h⁻¹. FIG. 8C shows yields at WHSV=0.3 h⁻¹. As seen from FIGS. 8A and 8B, selectivity to DVL was better at 2.7 h⁻¹ and 0.7 h⁻¹WHSV. At WHSV=0.3 h⁻¹, selectivity to DVL was markedly decreased. These three graphs taken together show that the relative concentrations of the compounds in the product mix can be tuned using the WHSV at which the reaction is conducted.

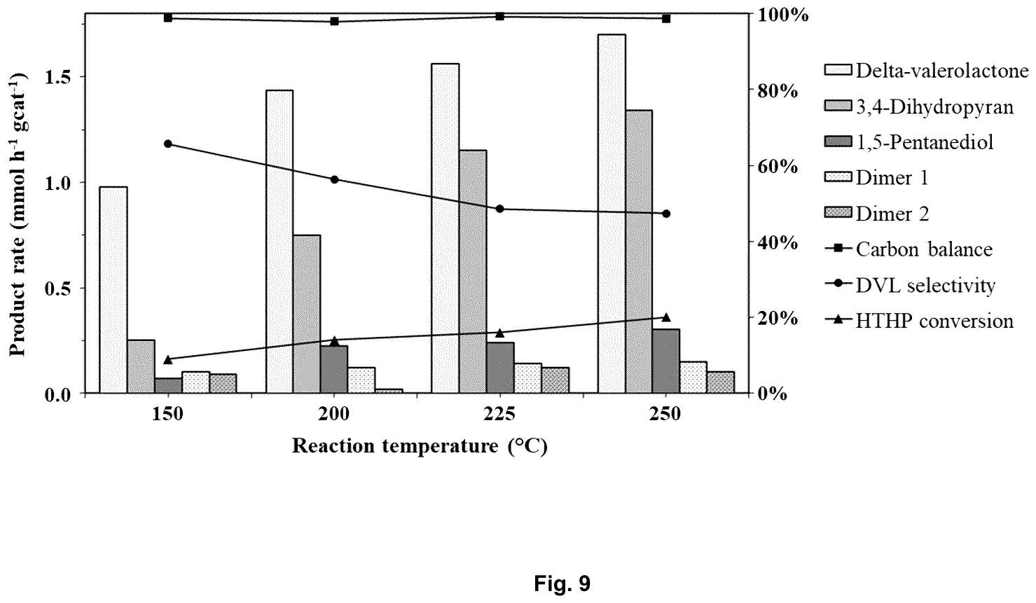

FIG. 9 is a graph showing production of DVL according to a specific version of the method disclosed and claimed herein. The reaction was conducted at 1 atm using a feedstock of HTHP in THP solution, fed into the reactor at a rate of 0.1 mL/min HTHP. The reactor was swept with a stream of argon at 100 mL/min Ar. The reactor contained 0.1 g of 20% Cu/SiO₂ catalyst. TOS was 24 h at each temperature with a fresh bed of catalyst for each run. The bars show product rate (mmol h⁻¹ g-cat⁻¹) (left-side Y-axis). Carbon balance (—▪—), DVL selectivity (—●—), and HTHP conversion (—▴—) are also shown (right-side Y-axis). The reaction yielded 66% selectivity to DVL at 150° C.

### EXAMPLES

### Example 1: Catalyst Screening for Converting HTHP to DVL

### Introduction

Global demand for high-performance polymers from the biomedical and pharmaceutical industries is projected to reach USD 54 billion by 2027. (Medical Plastics Market Size, Share & COVID-19 Impact Analysis, By Application, and Regional Forecast, 2020-2027. Fortune Business Insights. fortunebusinessinsights.com/medical-plastics-market-102136 (accessed 2023-10-02).) δ-Valerolactone (DVL) is a five-carbon (C5) cyclic carboxylic acid that undergoes ring-opening polymerization to poly(δ-valerolactone) (PVL)—a biocompatible polyester used in surgical bone grafts, tissue engineering scaffolds, and implantable drug delivery systems (IDDS). (See Badwelan, et al., Polymers 2020, 13 (1), 46; Zhang, et al. Advanced Materials 2020, 32 (50), 2005314; and Le Devedec, et al. Molecular Pharmaceutics 2018, 15 (4), 1565-1577, respectively.) Lactone/lactide-based polymers like PVL possess the unique property of in vivo biodegradability into nontoxic metabolites (lactic acid, CO₂, and water) via hydrolysis of the ester backbone in biological media. See Da Silva, et al. Chemical Engineering Journal 2018, 340, 9-14 and Hu, et al., Macromolecules 2018, 51 (7), 2526-2532. PVL also exhibits very high tensile strength and fracture strain, markedly outperforming commodity plastics like high density polyethylene (HDPE). (Li, et al. Angewandte Chemie International Edition 2023, e202303791.) Furthermore, PVL is chemically recyclable—DVL is recovered in quantitative yields by depolymerizing PVL with an acid catalyst at 100° C., with no loss in the properties of recycled PVL after 5 cycles—enabling closed loop PVL manufacturing. Li, et al. Nature Chemistry 2023, 15 (2), 278-285.

Herein, a novel route to DVL via dehydrogenation of biobased 2-hydroxytetrahydropyran (HTHP) over earth-abundant copper catalysts at selectivities >75% has been demonstrated. Unlike petroleum, lignocellulosic biomass is rich in C5 feedstock in the form of xylose—the second most abundant sugar in the world. Xylose dehydration is carried out for the production of furfural, with the furfural market projected to exceed 400,000 MT by 2024. (Furfural Market by Raw Material (Sugarcane Bagasse, Corncob, Rice Husk and Others), Application (Derivatives (Furfural Alcohol and Other Derivatives), solvent) and Region (Asia-Pacific, Americas, Europe,Middle East and Africa)—Global Forecast to 2024. marketsandmarkets.com/Market-Reports/furfural-market-101056456.html.) A 2-step pathway has been previously demonstrated from furfural-derived THFA to HTHP: i) Dehydration and Wagner-Meerwein rearrangement of THFA to 3,4-dihydropyran (DHP) over γ-Al₂O₃; and ii) Autocatalytic hydration DHP to HTHP. (See Li, et al., Applied Catalysis B: Environmental 2019, 245, 62-70 and Barnett, et al., ACS Sustainable Chemistry & Engineering 2017, 5 (11), 10223-10230, respectively. HTHP is an emerging platform chemical for the production of several C5 polymer precursors, including 1,5-pentanediol (PDO) and 5-amino-1-pentanol. (See Brentzel, et al., ChemSusChem 2017, 10 (7), 1351-1355 amd Li, et al., ACS Sustainable Chemistry & Engineering 2020, 8 (16), 6352-6362, respectively.) PDO production from hydrogenation of HTHP, as opposed to the fossil route, has been shown to have 95% reduction in greenhouse gas (GHG) emissions and 40-50% drop in the minimum selling price of PDO. (Huang, et al., ACS Sustainable Chemistry & Engineering 2017, 5 (6), 4699-4706.)

In this Example, numerous metal oxide and supported-transition metal catalysts were screened to elucidate the product profile from the thermocatalytic conversion of HTHP, and to determine which catalyst has the highest rates of DVL formation. Batch reactions were then carried out to reveal that HTHP is in thermal equilibrium with 3,4-dihydropyran (DHP), and two dimeric compounds. The rates of HTHP and DVL degradation to humins has also been studied. Next, the kinetics of DVL production over Cu/SiO₂ has been determined in a packed bed reactor at varying temperatures, contact times, and HTHP partial pressures. Finally, a kinetic model has been developed to model DVL selectivity as a function of both temperature and contact time.

### Materials and Methods

Materials: 3,4-Dihydropyran (Fisher Scientific, 99%), 2-hydroxytetrahydropyran (Acros Organics, 97%), δ-valerolactone (VWR International, 99%), 1,5-pentanediol (Thermo Scientific, 98%), tetrahydropyran (Sigma-Aldrich, 99%), Argon (Airgas, UHP grade), H₂ (Airgas, UHP grade), Cu(NO₃)₂·3H₂O (Sigma Aldrich), Ni(NO₃)₂·6H₂O (Fisher Scientific), Co(NO₃)₂·6H₂O (Fisher Scientific), SiO₂ (Sigma Aldrich), Al₂O₃ (Santa Cruz Biotechnology), MgO (Santa Cruz Biotechnology), Pd/SiO₂ (VWR International), and Pt/SiO₂ (Sigma Aldrich) were used as received. HTHP used in flow reactions was prepared via hydration of DHP and liquid-liquid extraction with THP.

Catalyst synthesis and characterization: SiO₂, Al₂O₃ and MgO catalysts were dried at 110° C. overnight in an air oven. Supported-metal catalysts containing 10 wt % of the desired metal were synthesized using incipient wetness impregnation of the desired metal salt into the support, followed by drying at 110° C. overnight in an air oven, then calcination at 460° C. in a muffle furnace (Thermo Fisher Thermolyne). Catalysts were reduced in situ in 100 mL/min H₂ flow at the required temperature before starting the reaction.

Batch reactor studies: Thermal studies with HTHP and DHP were carried out in 45 mL Hastelloy C-276 Parr reactor vessels, with each reactor head equipped with a PTFE flat gasket, K-type thermocouple, pressure transducer, safety rupture disk, manual inlet valve, exhaust valve, and dip tube for in situ product sampling. After addition of liquid feed and a stir bar, the reactors were sealed with 6 compression bolts, purged with Ar 3 times, then pressurized to 200 psi with Ar. Stirring was set to 800 RPM, and the reactor temperature was raised to the desired temperature. Vapor-phase products were sampled in 0.5 L Tedlar gas bags (Restek) attached to the reactor exhaust valve. At the end of each experiment, reactors were quenched in ice baths, depressurized and liquid-phase products were filtered through 0.2 μm syringe filters before analysis.

Continuous flow reactor studies: HTHP dehydrogenation to DVL was carried out in a fixed bed reactor consisting of ¼″ (6.35 mm) stainless steel tubing placed inside an aluminum heating block, which was then inserted into a programmable clamshell furnace (Thermo Fisher Lindberg Blue M). Liquid feed was pumped in using an HPLC pump (Eldex Optos Series) and Ar was co-fed using a mass flow controller (Brooks 5850E Series). Reactor tubes were packed with the desired mass of catalyst, with the catalyst bed held in place using glass beads and quartz wool on both ends. Supported-metal catalysts were reduced in situ in 100 mL/min H₂ at the desired temperature for 4 hours, then flushed with Ar and heated/cooled to the reaction temperature before each experiment. Reactor pressure was maintained at 200 psi using a spring-loaded back pressure regulator (Swagelok). Products were collected in a chilled cylindrical vessel maintained at 0° C. with a circulating water/ethylene glycol bath. Condensed products were sampled from the bottom of the collection vessel, while gas products were sent to an in-line gas chromatograph (GC).

Product analysis: Gas-phase products were analyzed with a Shimadzu GC-2014 equipped with a thermal conductivity detector (TCD) and flame ionization detector (FID). Liquid products were identified using a quadrupole gas chromatograph-mass spectrometer (Shimadzu GCMS-QP2010) and quantified using a Shimadzu GC-2010 equipped with an FID. External calibrations with known standards were carried out for the GC-TCD and GC-FIDs. Equations 3-4 are used to calculate the turnover frequency (TOF) and product selectivity, Equations 5-6 are used to determine the conversion of HTHP and the carbon balance, and Equation 7 is used for contact time.

$$Turnover frequency ( TOF , {min}^{- 1} ) = \frac{m {mol}_{product}}{{g}_{solution}} \cdot Flow rate ( \frac{{g}_{feed}}{min} ) \cdot \frac{1}{m {mol}_{Cu}} \qquad ( 3 )$$

$$Product selectivity ( \% ) = \frac{m {mol}_{product}}{m {mol}_{total products}} \cdot 100 \% \qquad ( 4 )$$

$$Conversion of HTHP ( \% ) = \frac{m {mol}_{HTHP , feed} - m {mol}_{HTHP , product}}{m {mol}_{HTHP , feed}} \cdot 100 \% \qquad ( 5 )$$

$$Carbon balance ( \% ) = ( \frac{m {mol}_{Carbon , feed}}{m {mol}_{Carbon , product}} ) \cdot 100 \% \qquad ( 6 )$$

$$Contact time ( min ) = \frac{{g}_{catalyst}}{{g}_{solution} / min} \qquad ( 7 )$$

### Results

### 1. HTHP Dehydrogenation to DVL—Catalyst Screening

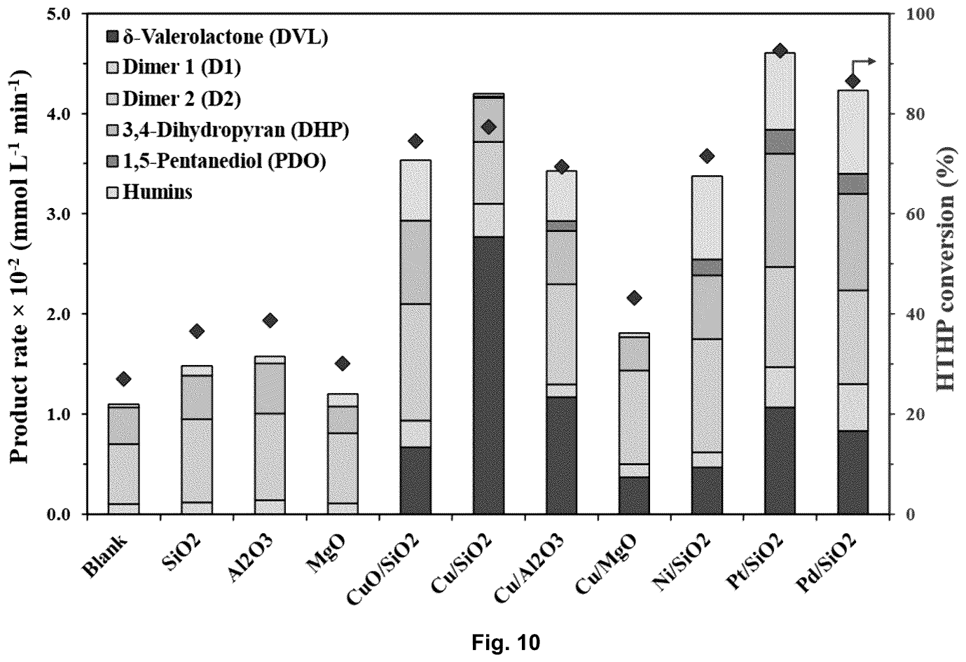

HTHP is an emerging platform chemical for the production of several C5 chemicals, but little is known about the thermal reactivity of HTHP under inert atmosphere. The reaction network observed from HTHP is shown in Scheme 1 and the product profiles over various catalysts are shown in FIG. 10. HTHP is observed to undergo 3 reactions irrespective of the catalyst used, if any: 1) Dehydration to 3,4-dihydropyran (DHP), 2) Acetalization to 2,2′-oxybis(tetrahydropyran) (Dimer 1; D1), 3) Acetalization to 5-(tetrahydropyran-2-yloxy)pentanal (Dimer 2; D2). Cyclic alcohols like cyclohexanol also undergo dehydration to their corresponding cycloalkenes but the reaction does not proceed without catalysts, unlike HTHP dehydration to DHP. (Bautista, et al. Applied Catalysis A: General 2003, 243 (1), 93-107.) HTHP is known to exhibit ring-chain tautomerism with 5-hydroxypentanal (5-HP), demonstrated using operando temperature-variable NMR.¹² For the purposes of this Example, HTHP/5-HP have been quantified as a single species as they are rapidly equilibrated over the GC column and elute as a single peak. In the absence of any catalysts, 27% of HTHP is converted to D2, DHP, and D1 at rates of 0.6, 0.36, and 0.1×10⁻² mmol L⁻¹ min⁻¹, respectively. Humins are also observed to form as dark-colored solids in the bottom of the reactor at 0.02 mM h⁻¹. Thermal treatment of biobased oxygenates can lead to humin formation due to random and uncontrollable condensation reactions. (Liu et al. Applications in Energy and Combustion Science 2022, 10, 100062.) Over SiO₂ and Al₂O₃, HTHP conversion is around 38%, with slightly higher rates of D2, DHP, and D1 formation. Over MgO, HTHP conversion drops to 30% and humins are formed 10 times faster (0.2 mM h⁻¹) than in the absence of catalysts.

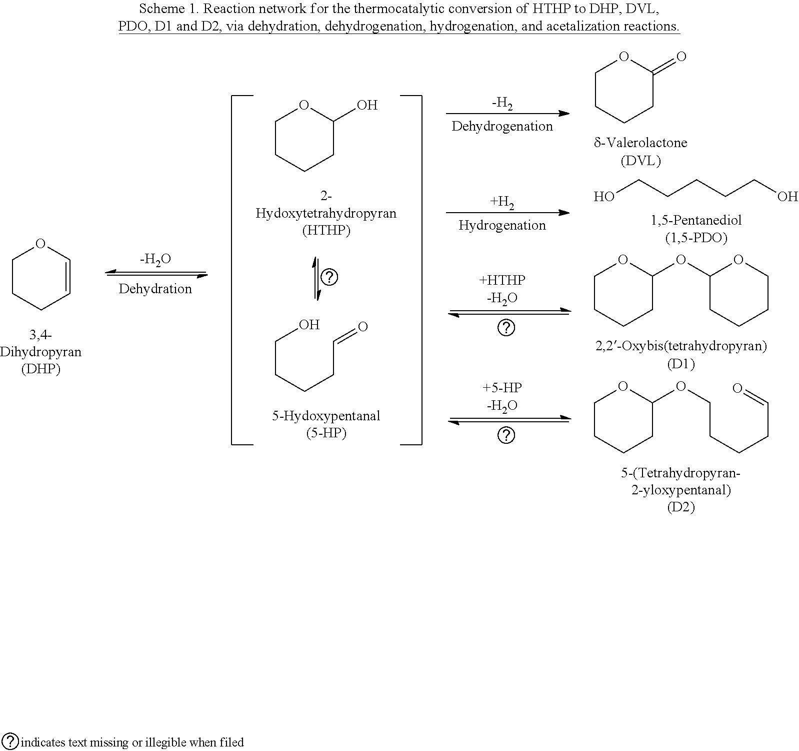

When a transition metal (Cu, Ni, Pt or Pd) is present in the catalyst, HTHP dehydrogenation to DVL occurs. All supported-metal catalysts except CuO/SiO₂ were reduced in situ and transferred to the reactor in an inert glovebox. DVL has the highest rate (1.6 mM h⁻¹) and highest selectivity (67%) over Cu/SiO₂ at these reaction conditions, corresponding to HTHP conversion of 77%. DVL production rates are observed to decrease in the following order: Cu/SiO₂>Cu/Al₂O₃>Pt/SiO₂>Pd/SiO₂>CuO/SiO₂>Cu/MgO. DVL rates over non-reduced CuO/SiO₂ are 4 times lower than over Cu/SiO₂ while humin formation is 20 times higher. Cu/SiO₂ has also been shown to be an excellent catalyst for cyclohexanol dehydrogenation to cyclohexanone. Fridman and Davydov, Journal of Catalysis 2000, 195 (1), 20-30.) HTHP hydrogenation to 1,5-pentanediol (PDO) from H₂ produced in situ (during HTHP dehydrogenation to DVL) is also observed over Ni/SiO₂, Cu/Al₂O₃, Pt/SiO₂, and Pd/SiO₂. While HTHP conversion over Pt/SiO₂ and Pd/SiO₂ are the highest out of all the catalysts studied, the selectivity to DVL is lower than over Cu/SiO₂ due to higher rates of DHP, PDO, and humin formation. Due to its superior activity in DVL production, Cu/SiO₂ will be used for kinetic studies in the rest of this Example.

2. Thermal equilibrium between HTHP, DHP, D1, and D2

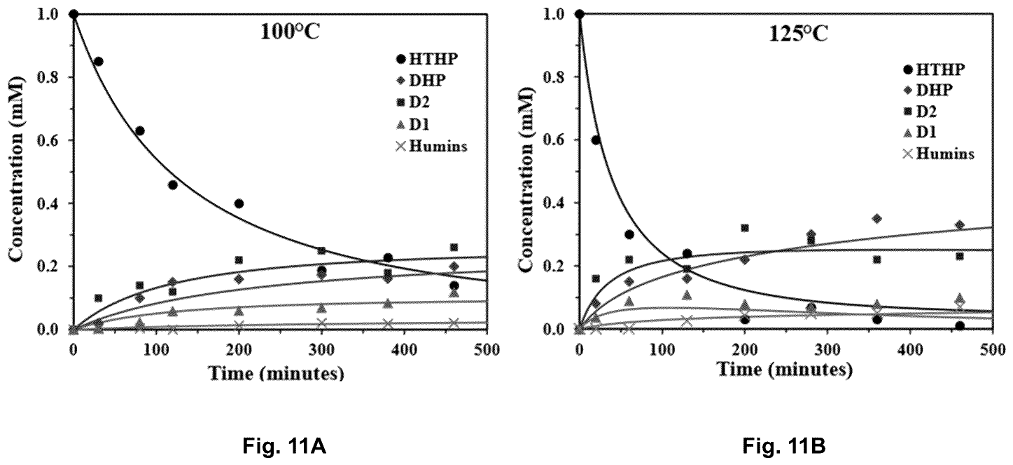

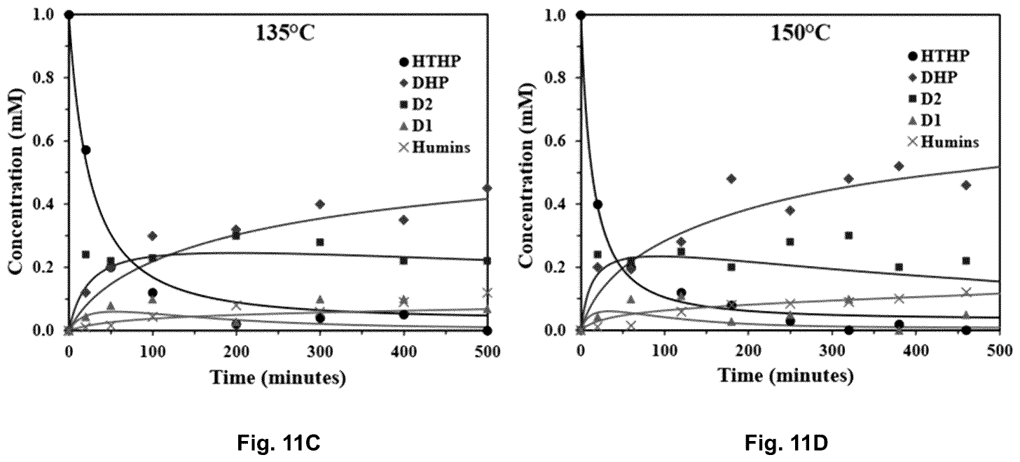

The concentration profiles of HTHP, DHP, D1, and D2 in the absence of any heterogeneous catalysts have been studied at 100-150° C. for 500 minutes to determine if these compounds are in thermal equilibrium (FIGS. 11A-11D). The fitted rate constants are listed in Table 5. At 100° C., 85% of HTHP is converted within 500 minutes to D2 (0.26 mM), DHP (0.2 mM), and D1 (0.12 mM). At 125° C., complete conversion of HTHP is achieved within 450 minutes, with DHP being the major product (0.35 mM), followed by D2 (0.23 mM) and D1 (0.1 mM). As the temperature is raised to 150° C., all HTHP is converted within just 300 minutes and the equilibrium composition of DHP reaches 0.45 mM, which is nearly twice that of D2 (0.2 mM) and five times higher than that of D1 (0.2 mM). The carbon balance is ˜99% at 100° C., but the formation of humins is observed at T>125° C. The rate of humin formation rises monotonically with temperature, with 0.12 mM of humins produced at 150° C. Previous studies have demonstrated that aqueous-phase hydration of DHP produces HTHP, with small amounds of D1 and D2 as byproducts.¹¹ This work is the first to demonstrate that DHP, HTHP, D1, and D2 are in thermal equilibrium. When HTHP is heated in the absence of water, the equilibrium is shifted back to DHP, D1, and D2.

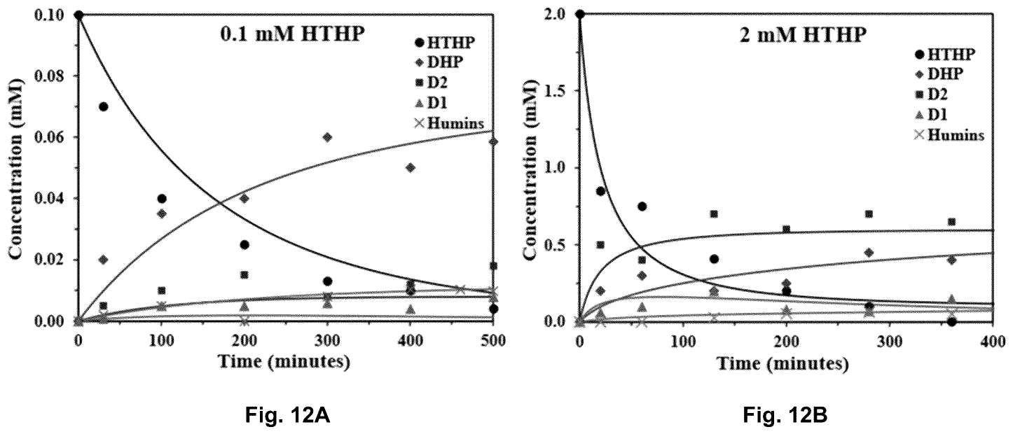

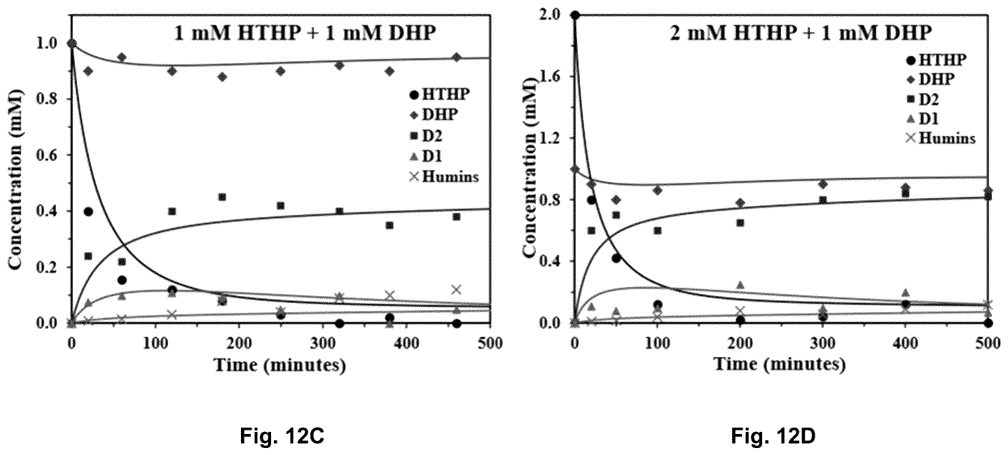

Next, experiments with variable feed compositions of HTHP and DHP were carried out to determine reaction orders, and to elucidate if D1 and D2 are formed from two molecules of HTHP dimerizing (with loss of a water molecule) or from DHP acetalizing with HTHP, or a combination of both. The results are shown in FIGS. 12A-12D and the fitted rate constants are listed in Table 5. No products are observed when the feed is pure DHP in the absence of HTHP or water. When the feed consists of 0.1 mM HTHP, DHP is the major product (0.06 mM) after 500 minutes. The study with 1 mM HTHP at 125° C. is shown above in FIG. 11B. Increasing the feed concentration to 2 mM HTHP leads to D2 being the major product (0.6 mM) within 350 minutes. Co-feed experiments with varying amounts of HTHP and DHP in the feed lead to DHP being the major component in the product mixture, followed by D2 then D1. The fitted model based on varying HTHP concentrations reveals that HTHP dehydration and humin formation are both 1^{st} order in HTHP concentration, while HTHP acetalization to D1 and D2 is 2^{nd} order. This study also reveals that D1 and D2 are forming from HTHP dimerization and from DHP acetalizing with HTHP.

TABLE 5
Fitted forward rate constants (k_{f}) and equilibrium constants (K_{eq})
of the thermal reactions between HTHP, DHP, D1, D2, and humins. Reaction
conditions: 100-150° C., 45 mL Parr reactor, 800 rpm, 200 psi Ar.

| | k_{f} (×10⁻³) ^{a} | | | | K_{eq} = k_{f}/k_{b} | | | |
|---|---|---|---|---|---|---|---|---|
| Reaction | 100° C. | 125° C. | 135° C. | 150° C. | 100° C. | 125° C. | 135° C. | 150° C. |
| R1: HTHP → DHP + H₂O | 1.2 | 4.0 | 5.6 | 9.9 | 1.6 | 7.1 | 10.5 | 11.2 |
| R2: 2HTHP → D1 + H₂O | 0.9 | 2.1 | 4.1 | 8.2 | 10.3 | 6.2 | 3.9 | 4.8 |
| R3: 2HTHP → D2 + H₂O | 2.3 | 5.3 | 11.8 | 23.1 | 72.1 | 78 | 78.2 | 80.1 |
| R4: HTHP + DHP → D1 | 0.9 | 2.2 | 3.4 | 4.9 | 4.8 | 1.1 | 0.5 | 0.2 |
| R5: HTHP + DHP → D2 | 1.6 | 4.3 | 5.9 | 10.3 | 13.5 | 12.1 | 6.8 | 4.1 |
| R6: HTHP → Humins | 0.1 | 0.6 | 1.2 | 2.5 | — | — | — | — |

^{a} The units of k_{f} are: min⁻¹ for R1, mmol L⁻¹ min⁻¹ for R2, R3, and R6, and (mmo² L⁻¹)² h⁻¹ for R4-R5; K_{eq} is unitless.

3. Reaction Mechanism for the Thermal Equilibrium Between HTHP, DHP, D1 and D2

Based on the kinetic studies carried out, the proposed reaction mechanism for the thermal equilibrium between HTHP, 5-HP, DHP, D1, and D2 is shown in Scheme 2. Ring-chain tautomerism of HTHP leads to 5-HP formation via intramolecular proton transfer between the ether and hydroxyl oxygen atoms. The rapid interconversion between the ring-open and ring-closed tautomers contributes to the high reactivity of the [HTHP/5-HP] feed. HTHP can also undergo protonation at the hydroxyl oxygen followed by dehydration to form a carbocation intermediate, which is resonance stabilized by the ether oxygen donating one of its lone pair of electrons to form a C═O bond within the ring. The proposed carbocation intermediate can then undergo two competing pathways: i) Deprotonation and C═C bond formation to produce DHP, or ii) Acetalization to D1 (or D2) by addition of HTHP (or 5-HP) and deprotonation at the central oxygen atom. When carrying out DHP hydration with water to produce HTHP (or when HTHP is co-fed with DHP), the vinyl carbon of DHP can abstract a proton from the feed mixture and re-form the carbocation intermediate, leading to the establishment of an equilibrium between DHP, HTHP, D1 and D2 once again. However, pure 20 DHP in the absence of a proton source does not react to form any products thermally

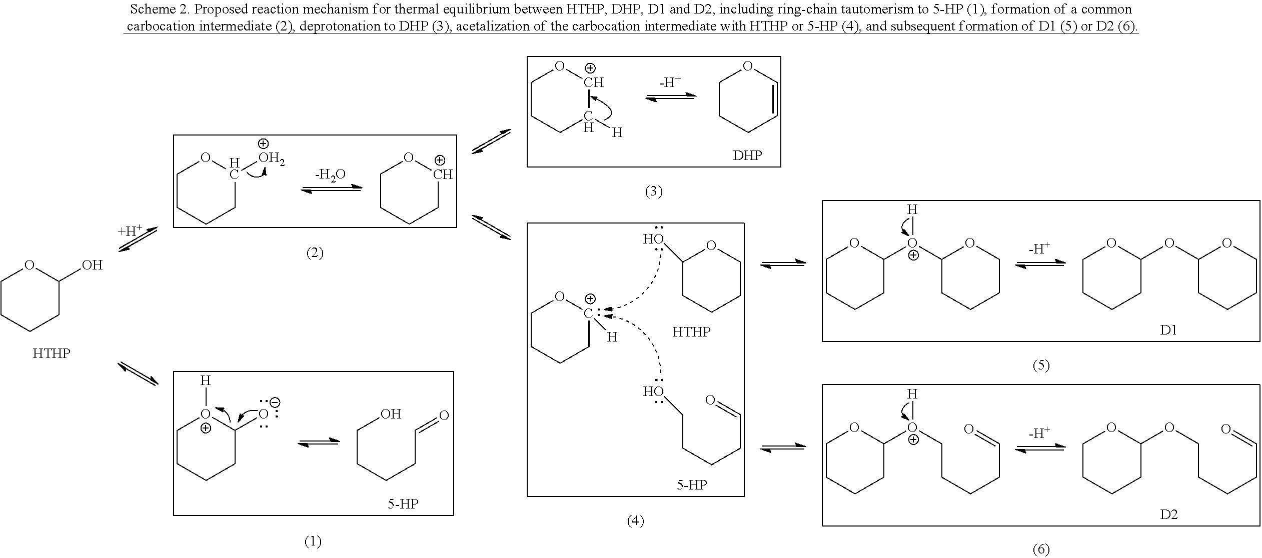

### 4. Effect of Temperature on Product Turnover Frequencies and Selectivities

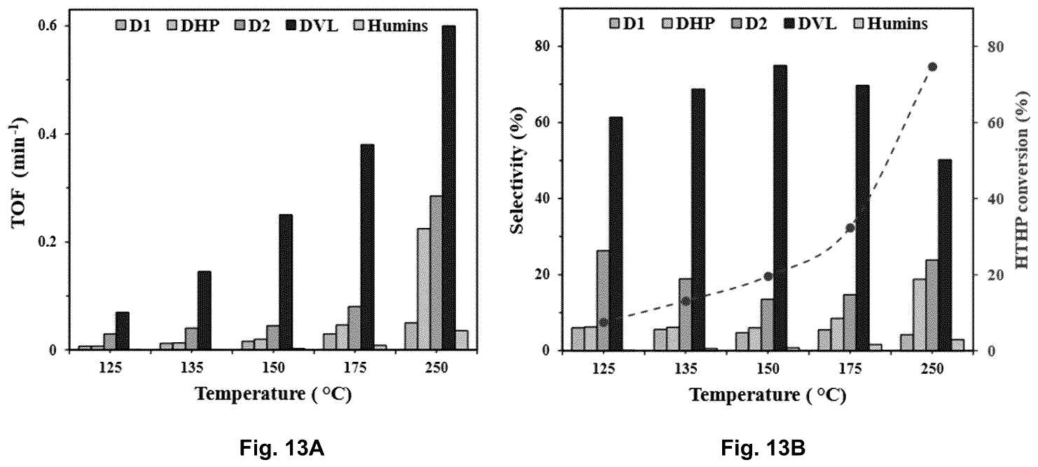

HTHP dehydrogenation to DVL over Cu/SiO₂ has been studied using a packed bed reactor to acquire kinetic data without loss of inert atmosphere over the catalyst. Cu/SiO₂ is chosen since the highest rates of DVL formation and selectivity to DVL have been observed over Cu/SiO₂. The effect of temperature on the turnover frequency (TOF) and selectivity of each product over Cu/SiO₂ is shown in FIGS. 13A and 13B. Reactions were carried out for 24 hours to obtain steady-state samples, and a fresh bed of Cu/SiO₂ was used for each temperature to rule out catalyst deactivation. Minimal HTHP conversion is observed at T<100° C., and the carbon balance is ˜98% for all experiments carried out at 125-250° C. The major product observed from HTHP over Cu/SiO₂ at all temperatures is DVL. At 125° C., HTHP conversion is only 7.5% with DVL produced at a TOF of 0.07 min⁻¹, corresponding to a selectivity of 61%. As the temperature is raised to 150° C., HTHP conversion increases to 19% while DVL formation jumps to 0.25 min⁻¹, reaching a peak DVL selectivity of 75.5%. Between 125-150° C., the selectivity to DHP and D1 remain nearly constant at 6.1 and 5.5%, respectively, while the selectivity to D2 drops from 26.3 to 13.6%. With further increase in temperature to 250° C., HTHP conversion reaches 77% but the selectivity to DVL drops to 39%. This is due to the sharp rise in DHP and D2 TOFs from 150-250° C., with DHP increasing 10× from 0.02 min⁻¹ to 0.22 min⁻¹, and D2 increasing 6× from 0.05 min⁻¹ to 0.3 min⁻¹. The apparent activation energies (Eₐ) and pre-exponential factors (A) of DVL, DHP, D1, and D2 formation have been determined using the Arrhenius equation from data collected at 125-175° C. within the kinetic regime (HTHP conversion <20%). The forward rate constants (k_{i,f}^{ref}) at reference temperature, T_{ref}=150° C., and the apparent activation energies (E_{a,i}) for each product are listed in Table 6. The Eₐ for HTHP hydration to DHP is observed to be 25 kJ mol⁻¹ lower than the Eₐ for DHP hydration to HTHP (78.8±1.5 kJ mol⁻¹),¹¹ albeit the latter was determined at 25-62.5° C. using 20 wt % DHP in water. The similarity in apparent activation energies for D1, D2, and DHP formation further corroborates the formation of a common carbocation intermediate from HTHP.

### 5. Effect of Contact Time on Product Turnover Frequencies and Selectivities

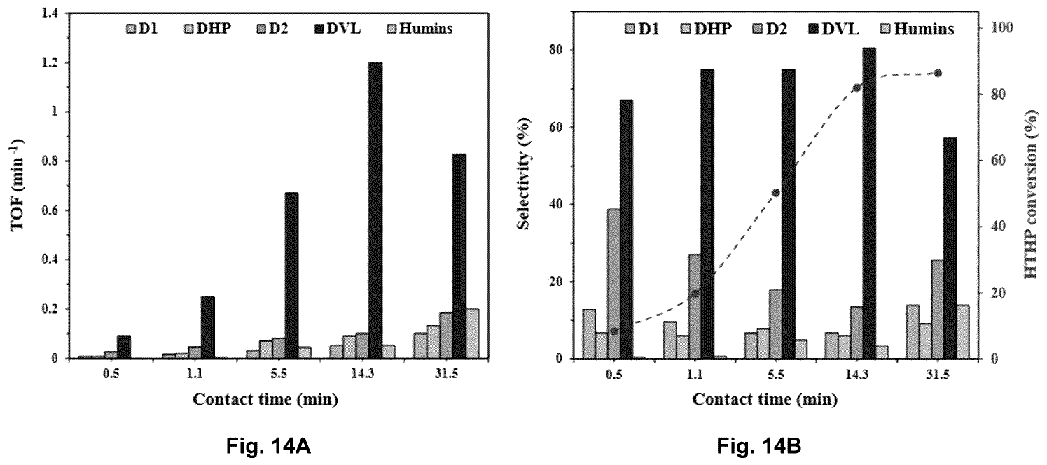

The effect of contact time, τ, on product turnover frequencies and selectivities was studied by varying the contact time at a fixed reaction temperature of 150° C. (FIGS. 14A and 14B). The TOF of DVL formations rises monotonically from 0.09 to 1 min⁻¹ as the contact time is increased from 0.5 to 14.3 minutes, but drops to 0.09 min⁻¹ with further increase in contact time to 31.5 min Correspondingly, the highest selectivity to DVL is observed to be 79% at 14.3 minutes. The temperature and contact time experiments herein reveal that intermediate contact times of ˜14.3 min at low temperatures of ˜150° C. are preferred to maximize selectivity to DVL.

6. Determining Reaction Orders from Partial Pressure Experiments

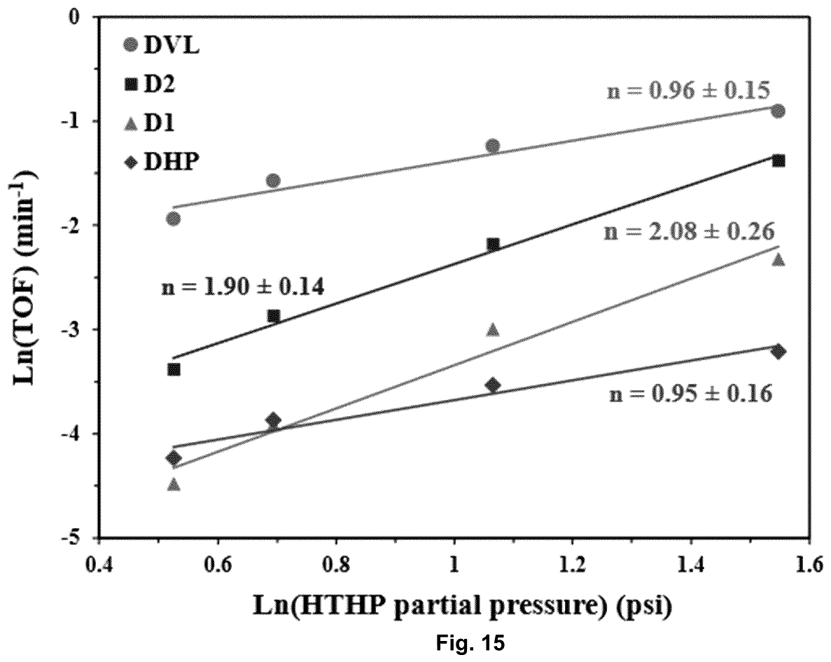

Reaction orders with respect to HTHP were determined by varying the HTHP partial pressure (PHTHP) from 1.7 to 4.7 psi, while keeping the temperature, contact time, and total pressure constant at 150° C., 1 min, and 200 psi, respectively. FIG. 15 shows the log-log plot of TOF versus P_{HTHP} along with the reaction orders (n) determined from the slope of each curve. As P_{HTHP} is increased from 1.7 to 4.7 psi, the TOF of DVL formation triples from 0.14 to 0.42 min⁻¹, while DHP quadruples from 0.01 to 0.04 min⁻¹. As seen from the slope of the DVL and DHP curves, HTHP dehydrogenation to DVL and HTHP dehydration to DHP are both nearly 1^{st} order in HTHP partial pressure. On the other hand, D1 and D2 rates increase 7× from 0.01 to 0.07 min⁻¹, and 0.04 to 0.28 min⁻¹, respectively, when P_{HTHP} is increased from 1.7 to 4.7 psi. Correspondingly, HTHP acetalization to D1 and D2 are both observed to be 2^{nd} order with respect to PHTHP.

### 7. Catalyst Stability as a Function of Time on Stream (TOS)

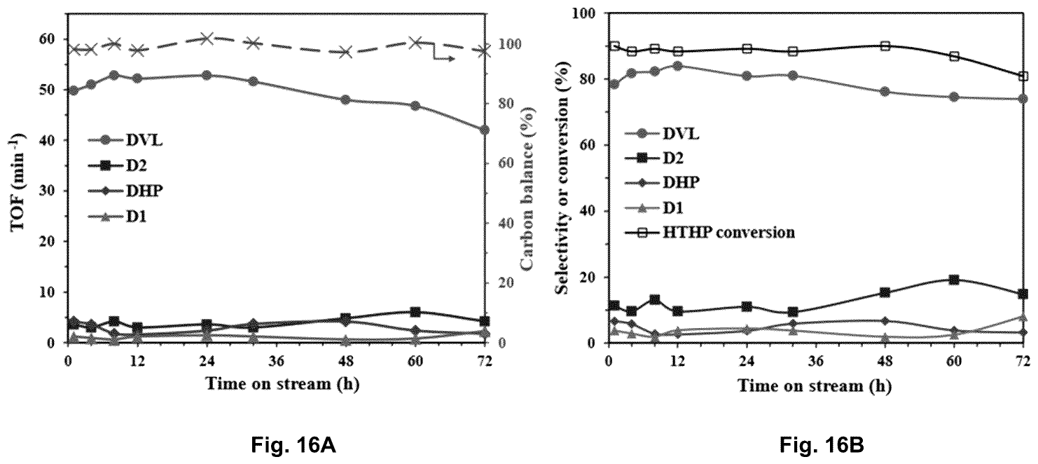

HTHP dehydrogenation over Cu/SiO₂ at the optimum reaction conditions found above (150° C. and 22 minutes of contact time) was carried out in a flow reactor for 72 h time on stream (TOS) to determine catalyst deactivation rates. As shown in FIGS. 16A and 16B, the TOF of DVL formation increased slightly from 49 to 52 min⁻¹ in the first 8 h then remained constant at ˜52 min⁻¹ till 32 h, indicating steady state had been achieved. The corresponding selectivity to DVL reached a maximum of 84% at 12 hours, which is slightly higher than model-predicted DVL selectivity at these conditions (81%). By 72 h, the TOF of DVL dropped to 42 min⁻¹ and the selectivity to DVL decreased to 74%. Similarly, HTHP conversion was ˜90% for 48 h but dropped to 81% by 72 h, indicating loss of catalyst activity. The TOF of D2, DHP, and D1 formation is <5 min⁻¹ at all times. But the selectivity to D1 and D2 increase to 8% and 19%, respectively, around 60 h. The loss in HTHP conversion and DVL rates, accompanied by the increase in rates of D1 and D2, indicated that the formation of C₁₀₊ species on the catalyst surface is contributing to catalyst deactivation via coking.

### 8. Development of a Reaction Kinetics Model

Based on the experiments carried out at varying reaction temperatures, contact times, and HTHP partial pressures, a kinetic model has been developed. The 8 reactions used in the model are shown in Scheme 3. The corresponding ordinary differential equations (ODEs) used in the kinetic model are listed in Equations 8-13. The forward rate constant for reaction i as a function of temperature, k_{f,i} (T), has been determined using the Arrhenius relationship in Equation 14, wherein k_{f,i}^{ref} is the experimentally determined forward rate constant at T_{ref}=423.15 K with apparent activation energy E_{a,i} (listed in Table 6). The backward rate constants a function of time, k_{b,i} (T), have been determined from Equation 15, wherein K_{eq,i}(T) is the equilibrium rate constant, which in turn is determined from ΔG_{rxn,i}(T).

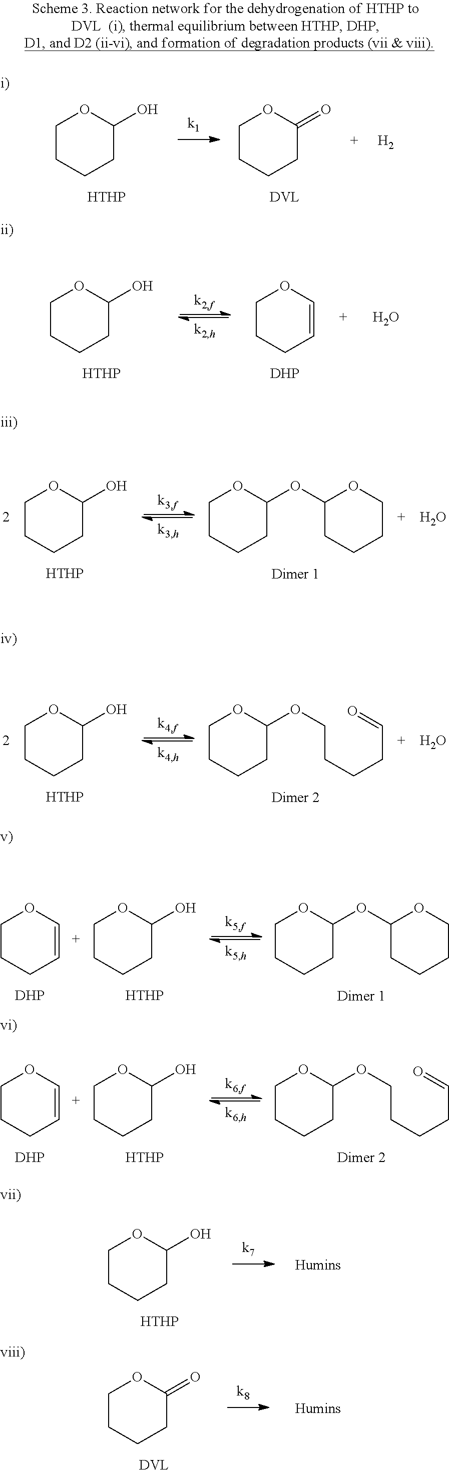

$$\frac{d [ DVL ]}{d τ} = {k}_{1} [ HTHP ] - {k}_{8} [ DVL ] \qquad ( 8 )$$

$$\frac{d [ HTHP ]}{d τ} = - {k}_{1} [ HTHP ] - {k}_{2 , f} [ HTHP ] + {k}_{2 , b} [ DHP ] [ {H}_{2} O ] - {{k}_{3 , f} [ HTHP ]}^{2} + {k}_{3 , b} [ D 1 ] [ {H}_{2} O ] - {{k}_{4 , f} [ HTHP ]}^{2} + {k}_{4 , b} [ D 2 ] [ {H}_{2} O ] - {k}_{5 , f} [ HTHP ] [ DHP ] + {k}_{5 , b} [ D 1 ] - {k}_{6 , f} [ HTHP ] [ DHP ] + {k}_{6 , b} [ D 2 ] \qquad ( 9 )$$

$$\frac{d [ DHP ]}{d τ} = {k}_{2 , f} [ HTHP ] - {k}_{2 , b} [ DHP ] [ {H}_{2} O ] - {k}_{5 , f} [ HTHP ] [ DHP ] + {k}_{5 , b} [ D 1 ] - {k}_{6 , f} [ HTHP ] [ DHP ] + {k}_{6 , b} [ D 2 ] \qquad ( 10 )$$

$$\frac{d [ D 1 ]}{d τ} = {{k}_{3 , f} [ HTHP ]}^{2} - {k}_{3 , b} [ D 1 ] [ {H}_{2} O ] + {k}_{5 , f} [ HTHP ] [ DHP ] - {k}_{5 , b} [ D 1 ] \qquad ( 11 )$$

$$\frac{d [ D 2 ]}{d τ} = {{k}_{4 , f} [ HTHP ]}^{2} - {k}_{4 , b} [ D 2 ] [ {H}_{2} O ] + {k}_{6 , f} [ HTHP ] [ DHP ] - {k}_{6 , b} [ D 2 ] \qquad ( 12 )$$

$$\frac{d [ Deg ]}{d τ} = {k}_{7} [ HTHP ] - {k}_{8} [ DVL ] \qquad ( 13 )$$

$${k}_{f , i} ( T ) = {k}_{f , i}^{ref} exp [ - \frac{{E}_{a , i}}{R} ( \frac{1}{T} - \frac{1}{{T}_{ref}} ) ] \qquad ( 14 )$$

$${k}_{b , i} ( T ) = \frac{{k}_{f , i} ( T )}{{K}_{eq , i} ( T )} \qquad ( 15 )$$

$$where {K}_{eq , i} ( T ) = exp ( - \frac{Δ {G}_{rxn , i} ( T )}{R \cdot T} ) \qquad ( 16 )$$

TABLE 6
Model-predicted kinetic parameters (with associated 95% confidence intervals)
for the thermocatalytic reactions between HTHP, DVL, DHP, D1, D2, and humins in
a packed bed reactor over Cu/SiO₂. Reaction conditions: 125-150° C., 0.1 mL/min
10 wt % HTHP in THP, 50 mL/min Ar, 24 h, 0.1 g Cu/SiO₂.

| | Forward rate constants, k_{f, i}^{ref}, | | |
|---|---|---|---|
| | at reference temperature | | Apparent activation |
| Reaction | T_{ref} = 150° C. | | energy, E_{a, i} (kJ mol⁻¹) |
| R1: HTHP → DVL + H₂ | 0.11 ± 0.02 | min⁻¹ | 36.9 ± 1.3 |
| R2: HTHP → DHP + H₂O | 0.014 ± 0.002 | min⁻¹ | 48.5 ± 0.5 |
| R3: 2HTHP → D1 + H₂O | 0.005 ± 0.001 | L mmol⁻¹ min⁻¹ | 50.2 ± 0.7 |
| R4: 2HTHP → D2 + H₂O | 0.015 ± 0.006 | L mmol⁻¹ min⁻¹ | 51.7 ± 1.1 |
| R5: HTHP + DHP → D1 | 0.002 ± 0.0008 | L mmol⁻¹ min⁻¹ | 63.5 ± 1.3 |
| R6: HTHP + DHP → D2 | 0.003 ± 0.0005 | L mmol⁻¹ min⁻¹ | 68.9 ± 1.5 |
| R7: HTHP → Humins | 0.004 ± 0.001 | min⁻¹ | 40.2 ± 0.9 |
| R8: DVL → Humins | 0.02 ± 0.003 1 | min⁻¹ | 30.5 ± 0.4 |

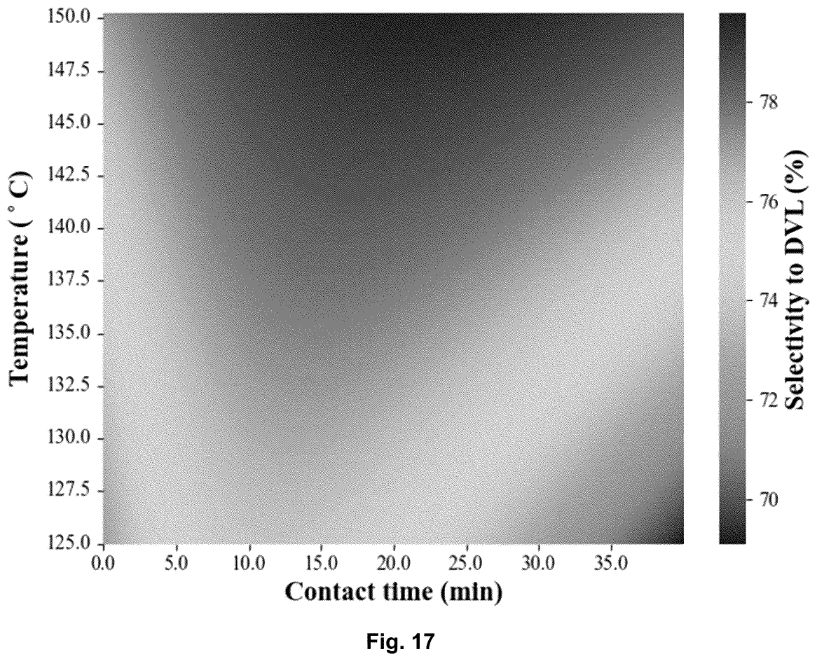

The resulting model-predicted selectivity to DVL as a function of temperature (125-150° C.) and contact time (0-40 minutes) is shown as a heatmap in FIG. 17. The model cannot be extrapolated to higher temperatures as the apparent activation energies would be very different from those listed in Table 6. At low temperatures, the rate of DVL production is too low, leading to lower selectivities. At long contact times, humin production increases, also leading to lower DVL selectivities. DVL selectivity can be maximized at 81% by carrying out HTHP dehydrogenation at a temperature of 150° C. and contact time of 22 minutes.

### Conclusions

We have demonstrated the catalytic production of δ-valerolactone (DVL) from dehydrogenation of biomass-derived 2-hydroxytetrahydropyran (HTHP) over Cu/SiO₂. We have also demonstrated for the first time that HTHP is in thermal equilibrium with 3,4-dihydropyran (DHP) via dehydration, and with 2,2′-oxybis(tetrahydropyran) (Dimer 1; D1) and 5-(tetrahydropyran-2-yloxy)pentanal (Dimer 2; D2) via acetalization. Using a series of experiments at varying temperatures, contact times, and feed compositions, we have developed a kinetic model consisting of 8 differential equations, wherein the forward (k_{f}) and backward (k_{b}) rate constants are scaled with respect to temperature using the Arrhenius equation and the equilibrium rate constant, K_{eq}. According to the model, the selectivity to DVL can be maximized at 81% by carrying out HTHP dehydrogenation over Cu/SiO₂ at 150° C. and 22 min of contact time. At lower temperatures and shorter contact times, DVL rates are very low. Conversely, at higher temperatures and longer contact times, degradation rates increase and humins are formed at higher rates, decreasing the selectivity to DVL. We have experimentally verified the model predictions and observed that the selectivity to DVL at the optimized reaction conditions can be as high as 84% at 12 hours time on stream.

### EXAMPLE 2: Production and Purification of HTHP via Hydration of DHP and Liquid-Liquid Extraction with THP

Because commercially available 2-hydroxytetrahydropyran (HTHP) is very expensive and we need a large volume of HTHP for continuous flow reactions, we prepared and purified HTHP in lab. First, we synthesized HTHP by hydrating 3,4-dihydropyran (DHP) with deionized (D. I.) water in a 20 wt % DHP solution in a 2 L batch reactor at 80° C. for 12 hours, according to previous method (U.S. Pat. No. 10,183,904). 100% conversion of DHP to HTHP was achieved. While DHP is immiscible in water, HTHP is miscible and remains in the aqueous solution. Before HTHP can be fed into the dehydrogenation reactor to produce 6-valerolactone (DVL), it needs to be separated from the remaining water in the product solution. Water in the feed solution leads to severe deactivation of the Cu/SiO₂ catalyst and degradation of DVL.

While the boiling points of water (100° C.) and HTHP (170° C.) are different, we cannot separate HTHP via distillation or rotary evaporation since HTHP is in thermal equilibrium with DHP and two dimeric compounds—2,2′-oxybis(tetrahydropyran) (Dimer 1; D1), and 5-(tetrahydropyran-2-yloxy)pentanal (Dimer 2; D2). Heating HTHP and removing water from the mixture leads to the equilibrium composition shifting back to DHP, D1, and D2, via dehydration and acetalization reactions. A physical separation at room temperature is required. We have achieved this by carrying out a liquid-liquid extraction (LLE) of HTHP in tetrahydropyran (THP) by pouring the aqueous solution of HTHP into a separatory funnel along with THP in a 1:1 (by mass) ratio. Water was removed from the bottom as water is denser than THP, while the HTHP-rich THP phase was obtained from the top. Any remaining water in the THP phase was removed by the addition of molecular sieves (hygroscopic aluminosilicate beads). The HTHP in THP solution was then fed to the dehydrogenation reactor.

THP was chosen due to the following seven reasons:
i. THP is chemically similar to HTHP (“like dissolves like”) and is an excellent solvent for HTHP.
ii. THP is easily separated from water at room temperature as THP has very low solubility in water (<8.5 wt %), unlike other ether solvents like tetrahydrofuran (THF) which are miscible in water.
iii. THP is chemically inert at the reaction conditions needed for HTHP dehydrogenation (150-300° C., inert atmosphere, Cu/SiO₂ catalyst) unlike other potential solvents like cyclohexanol. (Dastidar, et al. Green Chemistry 2022, 24 (23), 9101-9113.)
iv. THP has a boiling point (b.p.) of 88° C., which makes separation from DVL (b.p.: 219° C.) after the dehydrogenation reaction very convenient.
v. THP does not undergo ring-opening polymerization, which is vital because a major application of DVL is the production of polyesters via ring-opening polymerization of DVL.
vi. THP does not form explosive peroxides from autoxidation in air, unlike THF and other ethers, which enables the use of THP as the solvent without the addition of stabilizers like butylated hydroxytoluene (BHT), decreasing the separation/purification costs of DVL downstream.
vii. THP is produced via hydrogenation of DHP, and DHP can be produced from dehydration of biomass-derived tetrahydrofurfuryl alcohol (THFA). (U.S. Pat. No. 10,183,904.)

What is claimed is:

1. A method to make delta-valerolactone (DVL), the method comprising dehydrogenating 2-hydroxytetrahydropyran (“HTHP”) by contacting HTHP with a supported-metal catalyst for a time, at a temperature, and at a pressure wherein at least a portion of the HTHP is converted to DVL.

2. The method of claim 1, wherein the HTHP is neat.

3. The method of claim 1, wherein the HTHP is in an aqueous or an organic solution having a concentration of from about 5 wt % to about 99 wt % HTHP.

4. The method of claim 1, wherein the HTHP is in gas phase.

5. The method of claim 1, wherein the HTHP is contacted with the catalyst in the presence of an inert feed gas.

6. The method of claim 5, wherein the inert feed gas is selected from the group consisting of Ar, He, and N₂.

7. The method of claim 1, wherein the HTHP is contacted with the catalyst at a temperature of from about 100° C. to about 300° C.

8. The method of claim 1, wherein the HTHP is contacted with the catalyst at a pressure of from ambient atmospheric pressure to about 500 psi (about 3.5 MPa).

9. The method of claim 1, wherein the catalyst comprises a metal selected from the group consisting of Ti, V, Fe, Co, Cu, Ni, Mo, Rh, Ru, Pd, Au, Ag, Ir, Re, Pt, and combinations thereof.

10. The method of claim 1, wherein the catalyst is on or attached to a support selected from group consisting of carbon and oxides of Si, Al, Mg, and Al and combinations thereof.

11. The method of claim 1, wherein the HTHP is present in a feed mixture further comprising a compound selected from the group consisting of 5-hydroxypentanal, 2,2′-hydroxytetrahydropyran, tetrahydropyran-oxypentanal, and combinations thereof.

12. The method of claim 11, wherein the feed mixture comprises an equilibrium mixture of HTHP, 5-hydroxypentanal, 2,2′-hydroxytetrahydropyran, and tetrahydropyran-oxypentanal.

13. The method of claim 1, wherein the method yields a product mixture comprising DVL, dihydropyran, and 1,5-pentanediol.

14. The method of claim 1, wherein the HTHP is extracted from an aqueous solution, the extraction comprising:
adding tetrahydropyran (THP) to the aqueous solution to form a water phase and a THP phase, wherein the HTHP enters the THP phase; and
separating the water phase from the THP phase.

15. The method of claim 14, further comprising adding molecular sieves to the THP phase to remove remaining water in the THP phase.

16. The method of claim 15, wherein the molecular sieves comprise hygroscopic aluminosilicate beads.

17. The method of claim 14, wherein the THP is added to the aqueous solution in a mass ratio of 1:1 with HTHP.

18. A method to extract 2-hydroxytetrahydropyran (HTHP) from an aqueous solution, the method comprising:
adding tetrahydropyran (THP) to the aqueous solution to form a water phase and a THP phase, wherein the HTHP enters the THP phase; and
separating the water phase from the THP phase.

19. The method of claim 18, further comprising adding molecular sieves to the THP phase to remove remaining water in the THP phase.

20. The method of claim 19, wherein the molecular sieves comprise hygroscopic aluminosilicate beads.

21. The method of claim 18, wherein the THP is added to the aqueous solution in a mass ratio of 1:1 with HTHP.
